(19) United States
(12) Patent Application Publication
(10) Pub. No.: US 2026/0245696 A1
(43) Pub. Date: Aug. 20, 2026

(54) METHODS INCLUDING GEOMETRIC NETWORK ANALYSIS FOR SELECTING PATIENTS WITH HIGH GRADE SEROUS CARCINOMA OF THE OVARY TREATED WITH IMMUNE CHECKPOINT BLOCKADE THERAPY

(71) Applicants: Memorial Sloan-Kettering Cancer Center, New York, NY (US); Sloan-Kettering Institute for Cancer Research, New York, NY (US); Memorial Hospital for Cancer and Allied Diseases, New York, NY (US); The Research Foundation for the State University of New York, Albany, NY (US)

(72) Inventors: Larry Norton, New York, NY (US); Jung Hun Oh, New York, NY (US); Rena Elkin, New York, NY (US); Joseph Deasy, New York, NY (US); Allen Tannenbaum, Albany, NY (US)

(21) Appl. No.: 18/872,212

(22) PCT Filed: Jun. 6, 2022
(86) PCT No.: PCT/US2022/032320

§ 371 Date: Dec. 5, 2024

(51) Int. Cl. G16H 20/17; G16B 20/10; G16B 20/20; G16B 30/00; G16H 50/70

(52) U.S. Cl. CPC G16H 20/17; G16B 20/10; G16B 20/20; G16B 30/00; G16H 50/70

(57) ABSTRACT

The present disclosure provides gene network curvature-based methods, devices, and systems for determining whether a patient suffering from or diagnosed with ovarian cancer will benefit from immune checkpoint inhibitor therapy. The network curvature-based methods disclosed herein are based on copy number alterations (CNAs) at the gene level and provide an integrated measure of variability within cancer gene networks in ovarian cancer patients.

### TECHNICAL FIELD

The present technology relates to gene network curvature-based methods, devices, and systems for determining whether a patient suffering from or diagnosed with ovarian cancer will benefit from immune checkpoint inhibitor (ICI) therapy. The network curvature-based methods disclosed herein are based on copy number alterations (CNAs) at the gene level and provide an integrated measure of variability within cancer gene networks in ovarian cancer patients.

### BACKGROUND

The following description of the background of the present technology is provided simply as an aid in understanding the present technology and is not admitted to describe or constitute prior art to the present technology.

Biomarkers of response to immunotherapy in ovarian cancer remain underdeveloped. Unlike other cancers, high grade serous ovarian cancer (HGSOC) has not been shown to respond well to ICI and traditional biomarkers, such as TMB, have not been predictive in HGSOC. Additionally, in HGSOC to date, PD-L1 expression has unfortunately not been found to be predictive of response to ICI [D. Zamarin et al., J. Clinical Oncology, vol. 38, pp. 1814-1823, 2020; M. Disis et al., JAMA Oncology, vol. 5, pp. 393-401, 2019]. While the presence of tumor infiltrating lymphocytes (TILs) is prognostic in HGSOC [L. Zhang et al., New England J. Medicine, vol. 348, pp. 203-213, 2003] and other cancers, their predictive value for ICI response is questionable. In two published studies that have evaluated combination of ICI with PARP inhibitors in HGSOC, presence of TILs was not predictive of response [A. F″arkkil″a et al., Nature Communications, vol. 11, p. 1459, 2020; E. o. Lamperti, Clinical Cancer Research-Clinical Trials: Immunotherapy, vol. 11, p. 1459, 2020]. Thus, while these biomarkers have been predictive of response to ICI in other cancer types, their value in HGSOC is rather limited.

Accordingly, there is an urgent need for accurate methods for pre-selecting/identifying ovarian cancer patients that would be responsive to ICI therapy.

### SUMMARY OF THE PRESENT TECHNOLOGY

In one aspect, the present disclosure provides a method for selecting a patient suffering from or diagnosed with ovarian cancer for treatment with an immune checkpoint inhibitor therapy comprising: constructing a network comprising a plurality of nodes and edges representing a plurality of gene pathways, wherein each node of the network is a gene that is a component of a gene pathway and wherein each node is associated with a copy number alteration (CNA) metric, wherein the CNA metric for each node is based on genomic sequence data corresponding to at least one tumor of the patient; computing for the network an edge curvature for each of the plurality of edges; computing a node curvature for each of the plurality of nodes based on the edge curvatures of edges incident to each of the plurality of nodes; computing a total curvature metric of the network by determining a net node curvature over the plurality of nodes of the network; and selecting the patient for treatment with an immune checkpoint inhibitor therapy when the patient exhibits a total curvature metric satisfies a predetermined threshold. The ovarian cancer may be metastatic or primary. In some embodiments, the ovarian cancer is high grade serous ovarian cancer.

In certain embodiments, the immune checkpoint inhibitor therapy comprises one or more of an anti-PD-1 antibody, an anti-PD-L1 antibody, an anti-PD-L2 antibody, an anti-CTLA-4 antibody, an anti-TIM3 antibody, an anti-TIGIT antibody, an anti-VISTA antibody, an anti-B7-H3 antibody, an anti-BTLA antibody, an anti-CD73 antibody, or an anti-LAG-3 antibody. Additionally or alternatively, in certain embodiments, the immune checkpoint inhibitor therapy comprises one or more of pembrolizumab, nivolumab, cemiplimab, atezolizumab, avelumab, durvalumab, ipilimumab, tremelimumab, ticlimumab, JTX-4014, Spartalizumab (PDR001), Camrelizumab (SHR1210), Sintilimab (IB1I308), Tislelizumab (BGB-A317), Toripalimab (JS 001), Dostarlimab (TSR-042, WBP-285), INCMGA00012 (MGA012), AMP-224, AMP-514, KN035, CK-301, AUNP12, CA-170, or BMS-986189.

Additionally or alternatively, in some embodiments, the patient suffers from stage III or stage IV ovarian cancer. In certain embodiments, the patient exhibits high tumor mutation burden (TMB) or low TMB and/or is LST-high.

In any and all embodiments of the methods disclosed herein, the genomic sequence data is obtained by sequencing circulating tumor DNA (ctDNA) or sequencing DNA from a biopsied tumor isolated from the patient. In certain embodiments, the genomic sequence data is obtained via whole exome sequencing or targeted exome sequencing.

Additionally or alternatively, in some embodiments, the network is constructed using a protein-protein interactome (PPI) database. Examples of PPI databases include, but are not limited to, Human Reference Protein Database (HPRD), Human Reference Interactome (HuRI), or Search Tool for the Retrieval of Interacting Genes/Proteins (STRING). In some embodiments of the methods of the present technology, the plurality of nodes of the network comprises ABL1, ACTB, ACTG1, ACTN1, ACVR1, ADAM15, AKT1, AKT2, APP, AR, ARRB2, ATM, ATXN1, AXIN1, BCAR1, BCL2, BCL2L1, BRCA1, BTK, C14orfl, CASP8, CCND1, CCND3, CCNE1, CD247, CDC42, CDK4, CDK5, CDKN1A, CDKN1B, CHD3, COIL, COPS5, COPS6, CREBBP, CRK, CRMP1, CSNK2A2, CTNNB1, DAXX, DLG4, DNM2, DVL2, EGFR, EIF2AK2, EP300, ESR1, EWSR1, EZR, FASLG, FEZ1, FGFR1, FN1, FOS, FXR2, GFI1B, GNAI1, GRB2, GSK3B, HCK, HDAC3, HGS, HIPK2, HRAS, HSF1, HSP90AA1, HTT, IGF1R, INSR, ITGB1, JAK1, JAK2, JAK3, JUN, LYN, MAGEA11, MAP2K1, MAP3K7, MAPK1, MAPK14, MAPK8, MCM2, MCM7, MDF1, MDM2, MLLT4, MUC1, MYC, MYOC, NCOA2, NCOR1, NEDD4, NFKBIA, NINL, NOTCH1, NR3C1, NTRK1, PAK1, PARP1, PCNA, PDPK1, PIAS1, PIK3CA, PIK3R1, PIK3R2, PLCG1, PLG, PML, POLR2A, POU2F1, PPP2R5A, PRKCA, PRKCB, PRKCD, PRKCE, PRKCG, PRNP, PRSS23, PSEN1, PTK2, RAC1, RAD51, RAF1, RANBP9, RARA, RASA1, RB1, RBL1, RBPMS, RGS2, RHOA, RPA1, RUNX2, RXRG, SAT1, SH3KBP1, SHC1, SLC9A3R1, SMAD2, SMAD3, SMAD4, SMAD7, SMARCA4, SMURF1, SNAPIN, SRC, STAT1, SUMO1, SUMO2, SUMO4, SUV39H1, SVIL, SYK, SYN1, TGFBR1, TGFBR2, TGM2, TLE1, TP53, TRAF6, TRIP13, UBB, UPF1, UTP14A, VIM, WAS, XPO1, XRCC6, YAP1, YWHAE, and YWHAQ. In certain embodiments, the plurality of nodes of the network comprises ACTB, ACVR1, ADAM15, AKT1, APP, AR, ARRB2, ATXN1, AXIN1, BCL2, BRCA1, BTK, CASP8, CD247, CDC42, CDK5, CDKN1A, CHD3, COIL, COPS6, CREBBP, CRK, CRMP1, CSNK2A2, CTNNB1, DLG4, DVL2, EGFR, EIF2AK2, EP300, ESR1, EWSR1, FASLG, FGFR1, FN1, FXR2, GNAI1, GRB2, GSK3B, HDAC3, HGS, HIPK2, HRAS, HSF1, HSP90AA1, HTT, JAK1, JUN, LYN, MAGEA11, MAPK1, MAPK14, MDM2, MUC1, MYC, MYOC, NCOR1, NR3C1, NTRK1, PAK1, PARP1, PCNA, PDPK1, PIK3R1, PIK3R2, PLCG1, POLR2A, POU2F1, PPP2R5A, PRKCA, PRKCD, PRKCE, PTK2, RAC1, RAF1, RANBP9, RASA1, RB1, RHOA, RPA1, SHC1, SMAD2, SMAD3, SMAD4, SMAD7, SMARCA4, SMURF1, SNAPIN, SRC, STAT1, SUMO1, SUMO4, TGFBR1, TP53, UBB, VIM, XPO1, XRCC6, YWHAE, and YWHAQ.

Additionally or alternatively, in some embodiments of the methods disclosed herein, the edge curvature comprises an Ollivier-Ricci curvature. In certain embodiments, the edge curvature is further based on a weighted hop distance between two nodes associated with a respective edge, wherein the weighted hop distance is determined using node weights associated with the CNA metrics for the two nodes associated with the respective edge. The weighted hop distance may be indicative of a likelihood of interaction between the genes associated with the two nodes associated with the respective edge.

In any and all embodiments of the methods disclosed herein, the node curvature comprises a scalar curvature for a corresponding gene determined from a weighted sum of curvatures on all edges incident to the corresponding gene. In some embodiments, the total curvature metric comprises a net scalar curvature summed over all nodes of the network.

In another aspect, the present disclosure provides a system, comprising: one or more processors coupled to a non-transitory memory, the one or more processors configured to: (a) construct a network comprising a plurality of nodes and edges representing a plurality of gene pathways, wherein each node of the network is a gene that is a component of a gene pathway and wherein each node is associated with a copy number alteration (CNA) metric, wherein the CNA metric for each node is based on genomic sequence data corresponding to at least one tumor of the patient; (b) compute for the network an edge curvature for each of the plurality of edges; (c) compute a node curvature for each of the plurality of nodes based on the edge curvatures of edges incident to each of the plurality of nodes; (d) compute a total curvature metric of the network by determining a net node curvature over the plurality of nodes of the network; and (e) select the patient for treatment with an immune checkpoint inhibitor therapy when the patient exhibits a total curvature metric that is higher than a predetermined threshold.

### BRIEF DESCRIPTION OF THE DRAWINGS

FIG. 1. Curvature intuition on graphs. FIG. 1 shows that curvature is an intrinsic property of a surface, and therefore does not depend on how it is situated in space. For example, (b) the Earth is curved, (a) even though it appears flat when standing on its surface. Similarly, (c) a plane that is bent into an arc still has zero curvature. The apparent curvature is merely due to how it is embedded in space. Examples of canonical surfaces with zero, positive and negative curvature are shown, respectively, in (c), (d) and (e). Geodesic triangles can be used to determine the curvature of a surface without specifying its embedding. Compared to (Euclidean) flat space (c), fat (d) and skinny (e) triangles are characteristic of positive and negative curved spaces, respectively. Going from smooth surfaces to graphs, (f) a grid is analogous to a surface with zero curvature while (g) many triangles (indicative of redundancies or feedback mechanisms) are characteristic of graphs with positive curvature and (h) tree-like topologies (indicative of diverging paths) are characteristic of graphs with negative curvature. (i) On a graph, curvature between two nodes j and k is characterized by the ratio of the transport distance W₁(μⱼ,μₖ) between distributions μⱼ and μₖ (defined respectively on nodes j and k) and the underlying ground distance d(j,k) between the two nodes. The transport distance (W₁) comes from the theory of optimal mass transport (OMT) and provides a functional distance between the nodes that accounts for the shape of the distribution and amount of shared neighbors. Curvature is positive (resp., negative) when the transport distance (i.e., information) between nodes is smaller (resp., larger) than the ground distance between them, reflecting the ease with which information is shared between nodes.

FIG. 2. Weighted hop distance. FIG. 2 shows the weighted hop distances for every edge in Zachary's Karate Club Graph with all node weight values initialized equal to 1. The node color indicates if the corresponding person is a club officer (purple) or member (orange). The distance between edge-adjacent nodes is shown at the edge midpoint.

FIG. 3. Comparing graph metrics. FIG. 3 shows the distances between every two nodes in Zachary's Karate Club Graph, with all node weight values initialized equal to 1, are shown using (left) the hop distance and (right) weighted hop distance.

FIG. 4. Patient selection. FIG. 4 shows an outline of the patient selection process.

FIG. 5. Survival curves for HGSOC metastasis samples (n=32). FIG. 5 shows that the samples were stratified into low and high groups by the 25th percentile of total curvature and genomic parameters. P-values were derived from the log-rank test.

FIG. 6. Linear regression of total curvature onto clinical parameters using HGS samples (n=45). FIG. 6 shows that the lower triangle includes the Pearson correlation (r) and two-sided p-value for the hypothesis test with H₀: the slope is zero, using Wald test with t-distribution of the test statistic and 95% confidence interval.

FIG. 7. Genes involved in Serous Ovarian Cancer in the G-1 Phase of the Cell Cycle: The G-1 phase of the cell cycle can be divided up into cyclin D-cdk4/6 early events and cyclin E-cdk2 later events. FIG. 7 shows the inhibitors of these protein kinase activities, p38 and p16 for cyclin D and p53 and p21 for cyclin E are shown above the cyclin D and E panels. The activating pathways for cyclin D (TORC-1) and cyclin E (TORC-2) are shown below these panels. The mutational loss of TP53 and the amplification of cyclin E results in the loss of control of cyclin E levels and the hyper-amplification of centrosome numbers destabilizing the copy number control of chromosome numbers (aneuploidy) and gene copy numbers. Serous ovarian cancers commonly have K-RAS, MYC and AKT genes or chromosome amplifications and overexpression. The p21 gene is not mutated suggesting that it has additional functions required elsewhere for viability or that additional functions of p53 must be lost for ovarian cancers. Every gene highlighted in this figure can be found genetically altered in a cancer of other tissue types.

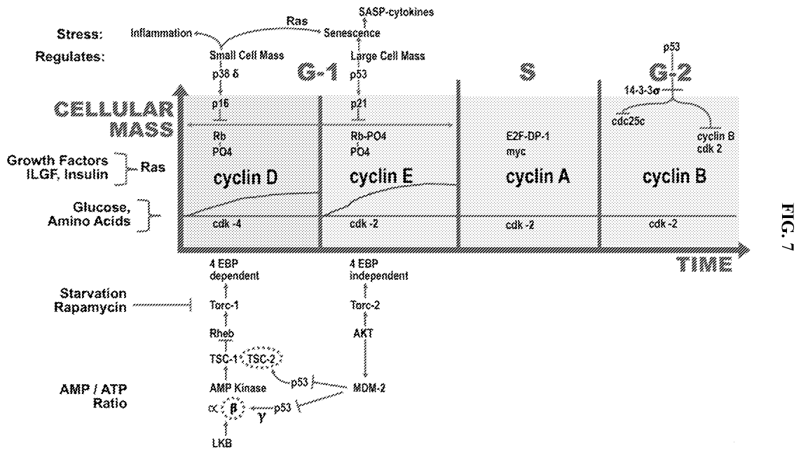

FIG. 8. K-fold cross-validation (K-CV). FIG. 8 shows that for HGS (n=45), 9-fold cross validated classification Kaplan-Meier curves and p-value determined from the empirical null distribution of the cross-validated logrank statistic. Briefly, the K-CV was performed as follows: the data set was partitioned into K groups of size n K where n is the total number of samples (here, n=45, K=9 and n K=5). The n K samples in the first partition were removed from the set. The cut for classification was determined to be the 25th percentile of the total curvature from the remaining n n/K samples. Each of the n K removed samples was then classified as low or high curvature by comparing their total curvature to the cutoff. The n K samples were then returned to the set and the process was repeated for each partition. This process terminated with each sample being classified by n n/K other samples resulting in two cross-validated high and low curvature groups. KM survival analysis was then performed between the predicted high and low risk groups and a log-rank statistic was computed. The 9-fold CV procedure was repeated 10,000 times. Out of the 10,000 tests, 39 trials resulted in cross-validated log-rank statistic greater than that of original data with test statistic=9.499, resulting in a significance level of 0.0039.

FIG. 9. Bootstrap validation. FIG. 9 shows the HGSOC (n=45) bootstrapped validation for KaplanMeier survival analysis. Bootstrap samples were generated by randomly selecting approximately 90% (n=40) for the HGSOC. The bootstrap sample was then stratified into high and low curvature groups by either (a) the bootstrap cutoff (the 25th percentile of the bootstrapped set's total curvature values) or (b) the fixed cutoff (the 25th percentile of the original set's total curvature values). In both cases, survival of each bootstrap group was estimated by the Kaplan-Meier analysis and the log-rank p-value was used to test if the survival curves for each group were different. P-values resulting from 1,500 iterations of this process are shown in increasing order by the blue lines. The dotted green lines emphasize the significance level at α=0.05. Out of the 1,500 trials, (a) 20 bootstrap-cutoff trials resulted in p>0.05 and (b) 15 fixed-cutoff trials resulted in p>0.05.

FIG. 10. Rationale for selecting 25th-percentile cutpoint. FIG. 10 shows the sorted total curvatures of all samples are shown by green circles. The dotted black line separates the low and high classified samples, where curvature is seen to start increasing slowly by the fitted line.

FIG. 11. Sub-curvature survival analysis. FIG. 11 shows survival curves for HGSOC patients (n=45) based on the 25th percentile of sub-curvature over the following subsets of genes: (a) all 171 curvature identified genes (FIG. 8) (b) 100 risk identified genes (FIG. 9).

FIG. 12. Network visualization. FIG. 12 shows a full resulting network taken as the largest connected component of the intersection between the HPRD and data (3,489 nodes, 9,710 edges, average degree=5.57) is shown in ForceAtlas 2 layout using Gephi. Top 171 identified genes are shown in red. Neighbors of the identified genes are shown in blue. Node size is scaled by degree.

FIG. 13. Subnetwork. FIG. 13 shows a subnetwork consisting of top 171 identified genes (red) and their neighbors (blue). This subnetwork has 2,084 nodes in total, 7,359 edges and average degree=7.06. Nodes are scaled by degree and the configuration was generated using Gephi's ForceAtlas 2 layout.

FIG. 14. Subnetwork. FIG. 14 shows a subnetwork consisting of top 171 identified genes. The 100 risk associated genes are shown in orange. Nodes are scaled by degree and the configuration was generated using Gephi's ForceAtlas 2 layout.

FIG. 15. Kaplan-Meier survival analysis of the Metabric dataset. FIG. 15 shows that the cohort (n=1903) was stratified into low and high groups by the 25th percentile of total curvature with the p-value derived from the log-rank test.

FIG. 16. Survival curves for HGSOC samples that did not receive ICIs. FIG. 16 shows HGSOC samples (IMPACT data, n=96) stratified into low and high groups by the 25th percentile of total curvature and available genomic parameters. Overall survival is defined as the duration from the time of diagnosis to death or time of last follow-up and p-values were derived from the log-rank test.

FIG. 17. Alternative cutpoint selection. FIG. 17 shows Kaplan-Meier survival analysis using alternative cutpoint for identifying high and low curvature groups with the maximally selected log-rank statistic (cutpoint=−13.62624, requiring 25% minimal proportion) using R's maxstat package. Note that this results in one sample being moved from the high curvature to the low curvature group as found by the 25th percentile of total curvature cutoff.

FIG. 18. Survival curves for HGSOC metastasis samples (n=32). FIG. 18 shows that the samples were stratified into low and high groups by the 25th percentile of total curvature and genomic parameters. P-values were derived from the log-rank test.

FIG. 19. FIG. 19 depicts an example system for curvature determination and patient selection in accordance with an illustrative embodiment.

FIG. 20. FIG. 20 depicts an example method for selecting a patient suffering from or diagnosed with ovarian cancer for treatment with an immune checkpoint inhibitor therapy.

FIG. 21. FIG. 21 is a block diagram of a server system and a client computer system in accordance with an illustrative embodiment.

### DETAILED DESCRIPTION

It is to be appreciated that certain aspects, modes, embodiments, variations and features of the present methods are described below in various levels of detail in order to provide a substantial understanding of the present technology.

The present disclosure is not to be limited in terms of the particular embodiments described in this application, which are intended as single illustrations of individual aspects of the disclosure. All the various embodiments of the present disclosure will not be described herein. Many modifications and variations of the disclosure can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled.

It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

In practicing the present methods, many conventional techniques in molecular biology, protein biochemistry, cell biology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

Genomic networks have a topology (i.e., a connectivity structure), but they also have a geometry, i.e., curvature, which gives a measure of their functional robustness. Graph curvature is intimately related to the number of invariant triangles, i.e., feedback loops at a given vertex, and the curvature between two vertices describes the degree of overlap between their respective neighborhoods [F. Bauer, J. Jost, and S. Liu, Math. Res. Lett., vol. 19, p. 11851205, 2012]. Informally, graphs (networks) with positive curvature characteristically contain many triangles (redundant feedback loops), contributing to its functional robustness with respect to a damaged or deleted edge. The more neighbors two given nodes have in common (i.e., triangles), the easier it is for information to flow between them. By weighing the ease with which information can be transferred from one node to another against the ground distance between them, curvature provides a local measure of functional connectivity compared to ordinary measures of connectivity which identify hubs based on degree. The present disclosure demonstrates that not only that the total curvature of a network can be used to predict overall patient survival in ovarian cancer, but it is also more effective than standard clinical parameters such as TMB.

Typically, the curvature is computed on a network using the standard hop distance (where every edge in a path connecting two nodes is treated as a hop) with node weights that are continuous in nature (e.g., gene expression). Here, a weighted hop distance derived from the data is used as the underlying graph metric, so the distance between two nodes depends not only on the topology, but on the likelihood of interaction as well. Using node weights assigned by (discrete) CNAs, the Examples show that curvature may also be informative in the discrete data setting. Furthermore, the Examples show that the network topology without any additional information may be used as a reference to identify potential key players responsible for the functional robustness, even when limited data is available, as demonstrated in this study.

Specifically, a shared topology with sample-specific gene interaction networks was created. The interactions may be obtained from a highly curated protein-protein interactome (PPI) database (e.g., Human Reference Protein Database (HPRD, [T. S. Keshava Prasad et al., Nucleic Acids Research, vol. 37, no. suppl 1, pp. D767-D772, 2009; S. Peri et al., Genome Research, vol. 13, no. 10, pp. 2363-2371, 2003]), The Human Reference Interactome (HuRI, [K. Luck et al., Nature, vol. 580, no. 7803, pp. 402-408, 2020]), Search Tool for the Retrieval of Interacting Genes/Proteins (STRING, [D. Szklarczyk et al., Nucleic Acids Research, vol. 47, no. D1, pp. D607-D613, 2019.]), where the protein interactions are assumed to serve as a proxy for the underlying gene interactions. The topology (i.e., connectivity) is then supplemented with sample-specific node weights which are based on copy number data. For each network, curvature is then computed at three scales: on edges, nodes, and the entire network. Analogous to Ricci curvature defined on tangent directions at a point on a Riemannian manifold and its contraction scalar curvature defined on the points of the manifold, the formulation of Ollivier-Ricci curvature is computed on all edges in the network and scalar curvature is computed on all nodes by contracting the OR (edge) curvature with the invariant distribution associated with the weighted network [Y. Ollivier, “Ricci curvature of markov chains on metric spaces,” J. Functional Analysis, vol. 256, pp. 810-864, 2009]. The total curvature of the network is then computed by contracting the scalar curvature to a single scalar. (See Eq. (9) for the precise definition.)

### Definitions

As it would be understood, the section or subsection headings as used herein is for organizational purposes only and are not to be construed as limiting and/or separating the subject matter described.

Unless defined otherwise, all technical and scientific terms used herein have the meaning commonly understood by a person skilled in the art to which this disclosure belongs. The following references provide one of skill with a general definition of many of the terms used in the present disclosure. Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise. The terminology used herein is for the purpose of describing particular embodiments only and is not intended to be limiting of the disclosure.

All numerical designations, e.g., pH, temperature, time, concentration, and molecular weight, including ranges, are approximations which are varied (+) or (−) by increments of 1.0 or 0.1, as appropriate or alternatively by a variation of +/−20% or +/−15%, or alternatively 10% or alternatively 5% or alternatively 2%. As will be understood by one skilled in the art, for any and all purposes, all ranges disclosed herein also encompass any and all possible subranges and combinations of subranges thereof. Furthermore, as will be understood by one skilled in the art, a range includes each individual member.

As used herein, the singular forms “a”, “an” and “the” are intended to include the plural forms as well, unless the context clearly indicates otherwise. For example, the term “a cell” includes a plurality of cells, including mixtures thereof.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value.

As used herein, the term “administration” of an agent to a subject includes any route of introducing or delivering the agent to a subject to perform its intended function. Administration can be carried out by any suitable route, including, but not limited to, intravenously, intramuscularly, intraperitoneally, subcutaneously, and other suitable routes as described herein. Administration includes self-administration and the administration by another.

The terms “cancer” or “tumor” are used interchangeably and refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell. As used herein, the term “cancer” includes premalignant, as well as malignant cancers. In some embodiments, the cancer is ovarian cancer.

As used herein, a “chromosome” refers to a discrete threadlike structure of nucleic acids and proteins that carries genetic information in the form of genes. Chromosomes are visible as morphological entities only during cell division. In humans, each chromosome has two arms, the p (short) arm and the q (long) arm. The short and long chromosome arms are separated from each other only by a centromere, which is the point at which the chromosome is attached to the mitotic spindle during cell division. A chromosome contains roughly equal parts of protein and DNA. The chromosomal DNA contains an average of 150 million nucleotides or bases. The 3 billion base pairs in the human genome are organized into 24 chromosomes. All genes are arranged linearly along the chromosomes. Generally the nucleus of a human cell contains two sets of chromosomes: a maternal set and a paternal set. Each set has 23 single chromosomes: 22 autosomes and an X or a Y sex chromosome.

As used herein, “chromosome gain” refers to the duplication of a chromosome or a chromosomal segment (e.g., p (short) arm or q (long) arm) leading to an unbalanced chromosome complement, or any chromosome number that is not an exact multiple of the haploid number (which is 23 in humans).

As used herein, “chromosome loss” refers to the loss of a chromosome or a chromosomal segment (e.g., p (short) arm or q (long) arm) leading to an unbalanced chromosome complement, or any chromosome number that is not an exact multiple of the haploid number (which is 23 in humans).

As used herein, the terms “copy number alteration” and “CNA” are used interchangeably and refer to somatic changes to chromosome structure that result in gain or loss in copies of sections of DNA, and are prevalent in many types of cancer. CNAs involve larger portions of the genome that can either be lost (deletions) or duplicated (amplifications) that may or may not contain a gene(s) with a size as low as a few kilobases up to entire chromosomes. Tumors in different patients carry variable amounts of these deletions or amplifications, which together are known as the CNA burden.

“Curvature” is a local measure of how a geometric object (e.g., curve, surface, space) deviates from being flat in the Euclidean sense. As used herein, a change in curvature refers to a difference in curvature between networks.

As used herein, “curvature of the edges of a network” refers to a parameter that measures the changes in the architecture or connectivity of a network of gene pathways for each individual tumor, where each node represents a gene and is quantitated by CNAs. Curvature measures the connectivity in the sense of feedback loops, and the copy number measures the abundance of each node and its projected impact upon the changes in the network architecture. In some embodiments, nodal curvature exhibits more variation than the CNAs, reflecting the integration of the gene copy numbers and the local impact of their alteration on the network.

As used herein, a “deletion” refers to a mutation (or a genetic alteration) in which part of a DNA sequence at a chromosome location is absent or lost compared to that observed in a reference genome. A deletion may occur within a gene or may encompass one or more genes. A “homozygous deletion” refers to the loss of both alleles of a gene within a genome. A homozygous deletion may comprise a partial or complete loss of each copy (maternal and paternal) of the gene sequence.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a composition known to exhibit the desired therapeutic effect) and a negative control (a subject or a sample that does not receive the therapy or receives a placebo) are typically employed.

As used herein, an “edge” of a network refers to one of the connections between two nodes (or vertices) of the network. In some embodiments, the edges are unidirectional or bidirectional.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression can include splicing of the mRNA in a eukaryotic cell. The expression level of a gene can be determined by measuring the amount of mRNA or protein in a cell or tissue sample. In one aspect, the expression level of a gene from one sample can be directly compared to the expression level of that gene from a control or reference sample. In another aspect, the expression level of a gene from one sample can be directly compared to the expression level of that gene from the same sample following administration of the compositions disclosed herein. The term “expression” also refers to one or more of the following events: (1) production of an RNA template from a DNA sequence (e.g., by transcription) within a cell; (2) processing of an RNA transcript (e.g., by splicing, editing, 5′ cap formation, and/or 3′ end formation) within a cell; (3) translation of an RNA sequence into a polypeptide or protein within a cell; (4) post-translational modification of a polypeptide or protein within a cell; (5) presentation of a polypeptide or protein on the cell surface; and (6) secretion or presentation or release of a polypeptide or protein from a cell.

As used herein, “fraction of the genome altered” or “FGA” refers to the percentage of copy number altered regions out of all sequenced regions. In some embodiments, FGA may be defined as the number of bases in sequenced genomic segments with log 2 copy number fold change >0.2 or <−0.2 over the total number of bases in all sequenced segments.

“Gene” as used herein refers to a DNA sequence that comprises regulatory and coding sequences necessary for the production of an RNA, which may have a non-coding function (e.g., a ribosomal or transfer RNA) or which may include a polypeptide or a polypeptide precursor. The RNA or polypeptide may be encoded by a full length coding sequence or by any portion of the coding sequence so long as the desired activity or function is retained. Although a sequence of the nucleic acids may be shown in the form of DNA, a person of ordinary skill in the art recognizes that the corresponding RNA sequence will have a similar sequence with the thymine being replaced by uracil, i.e., “T” is replaced with “U.”

As used herein, “gene amplification” refers to an increase in the number of partial or complete copies of a single gene sequence or several gene sequences at a specific chromosome locus without a proportional increase in other genes. In some embodiments, gene amplifications can result from duplication of a DNA segment that contains a gene through errors in DNA replication and repair machinery. Gene amplification is common in cancer cells, and may cause an increase in the corresponding RNA and protein encoded by the amplified gene(s).

As used herein, “haploid” describes a cell that contains a single set of chromosomes, e.g., a copy of each autosome and one sex chromosome. In humans, gametes are haploid cells that contain 23 chromosomes, each of which represents one of a chromosome pair that exists in diploid cells. The number of chromosomes in a single set is represented as n, which is also called the haploid number (In humans, n=23).

As used herein, “immune checkpoint inhibitor therapy” refers to a form of cancer immunotherapy that targets immune checkpoints, key regulators of the immune system, that when stimulated can dampen the immune response to an immunologic stimulus. Some cancers evade the immune system by stimulating immune checkpoint targets. Checkpoint therapy can block inhibitory checkpoints, restoring immune system function. Examples of immune checkpoint inhibitors include, but are not limited to, an anti-PD-1 antibody, an anti-PD-L1 antibody, an anti-PD-L2 antibody, an anti-CTLA-4 antibody, an anti-TIM3 antibody, an anti-TIGIT antibody, an anti-VISTA antibody, an anti-B7-H3 antibody, an anti-BTLA antibody, an anti-CD73 antibody, or an anti-LAG-3 antibody.

As used herein, “Large-scale state scores” or “LST” is defined as a chromosomal breakpoint resulting in allelic imbalance between adjacent regions of at least 10 Mb. In some embodiments, a high LST refers to a chromosomal breakpoint resulting in allelic imbalance between adjacent regions of >15 Mb.

As used herein, a “mutation” of a gene refers to the presence of a variation within the gene or gene product that affects the expression and/or activity of the gene or gene product as compared to the normal or wild-type gene or gene product. The genetic mutation can result in changes in the quantity, structure, and/or activity of the gene or gene product in a cancer tissue or cancer cell, as compared to its quantity, structure, and/or activity, in a normal or healthy tissue or cell (e.g., a control). For example, a mutation can have an altered nucleotide sequence (e.g., a mutation), amino acid sequence, expression level, protein level, protein activity, in a cancer tissue or cancer cell, as compared to a normal, healthy tissue or cell. Exemplary mutations include, but are not limited to, point mutations (e.g., silent, missense, or nonsense), deletions, insertions, inversions, linking mutations, duplications, translocations, inter- and intra-chromosomal rearrangements. Mutations can be present in the coding or non-coding region of the gene. In certain embodiments, the mutations are associated with a phenotype, e.g., a cancerous phenotype (e.g., one or more of cancer risk, oncogenesis, immunogenicity, or responsiveness to treatment). In one embodiment, the mutation is associated with one or more of: a genetic risk factor for cancer, a positive treatment response predictor, a negative treatment response predictor, a positive prognostic factor, a negative prognostic factor, or a diagnostic factor. As used herein, a “missense mutation” refers to a mutation in which a single nucleotide substitution alters the genetic code in a way that produces an amino acid that is different from the usual amino acid at that position. In some embodiments, missense mutations alter one or more functions or physical-chemical properties of the encoded protein.

As used herein, a “network” refers to a plurality of nodes that are connected by edges. Networks can represent many different types of data. A “biological network” is a representation of systems as complex sets of binary interactions or relations between various biological entities. In general, networks or graphs are used to capture relationships between entities or objects. In some embodiments, the nodes of the network represent different entities (e.g. proteins or genes in biological networks), and edges convey information about the connections between the nodes.

As used herein, a “node” represents an intersection point or vertex of a network.

The terms “polynucleotide”, “nucleic acid” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this disclosure that is a polynucleotide encompasses both the double-stranded form and each of two complementary single-stranded forms known or predicted to make up the double-stranded form. A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

The terms “polypeptide,” “peptide,” and “protein” are used interchangeably herein to refer to a polymer of amino acid residues. The terms apply to naturally occurring amino acid polymers as well as amino acid polymers in which one or more amino acid residues are a non-naturally occurring amino acid, e.g., an amino acid analog. The terms encompass amino acid chains of any length, including full length proteins, wherein the amino acid residues are linked by covalent peptide bonds.

As used herein, the term “sample” refers to clinical samples obtained from a subject. In certain embodiments, a sample is obtained from a biological source (i.e., a “biological sample”), such as tissue, bodily fluid, or microorganisms collected from a subject. Sample sources include, but are not limited to, mucus, sputum, bronchial alveolar lavage (BAL), bronchial wash (BW), whole blood, bodily fluids, cerebrospinal fluid (CSF), urine, plasma, serum, or tissue.

As used herein, the terms “subject,” “individual,” or “patient” are used interchangeably and refer to an individual organism, a vertebrate, or a mammal and may include humans, non-human primates, rodents, and the like (e.g., which is to be the recipient of a particular treatment, or from whom cells are harvested). In certain embodiments, the individual, patient or subject is a human.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. Therapeutic effects of treatment include, without limitation, inhibiting recurrence of disease, alleviation of symptoms, diminishment of any direct or indirect pathological consequences of the disease, preventing metastases, decreasing the rate of disease progression, amelioration or palliation of the disease state, and remission or improved prognosis. By “treating a cancer” is meant that the symptoms associated with the cancer are, e.g., alleviated, reduced, cured, or placed in a state of remission.

It is also to be appreciated that the various modes of treatment of diseases as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

### Curvature-Based Methods and Systems of the Present Technology

In one aspect, the present disclosure provides a method for selecting a patient suffering from or diagnosed with ovarian cancer for treatment with an immune checkpoint inhibitor therapy comprising: constructing a network comprising a plurality of nodes and edges representing a plurality of gene pathways, wherein each node of the network is a gene that is a component of a gene pathway and wherein each node is associated with a copy number alteration (CNA) metric, wherein the CNA metric for each node is based on genomic sequence data corresponding to at least one tumor of the patient; computing for the network an edge curvature for each of the plurality of edges; computing a node curvature for each of the plurality of nodes based on the edge curvatures of edges incident to each of the plurality of nodes; computing a total curvature metric of the network by determining a net node curvature over the plurality of nodes of the network; and selecting the patient for treatment with an immune checkpoint inhibitor therapy when the patient exhibits a total curvature metric satisfies a predetermined threshold. The ovarian cancer may be metastatic or primary. In certain embodiments, the ovarian cancer is high grade serous ovarian cancer.

Additionally or alternatively, in some embodiments, the patient suffers from stage III or stage IV ovarian cancer. The patient may exhibit high tumor mutation burden (TMB) or low TMB and/or is LST-high.

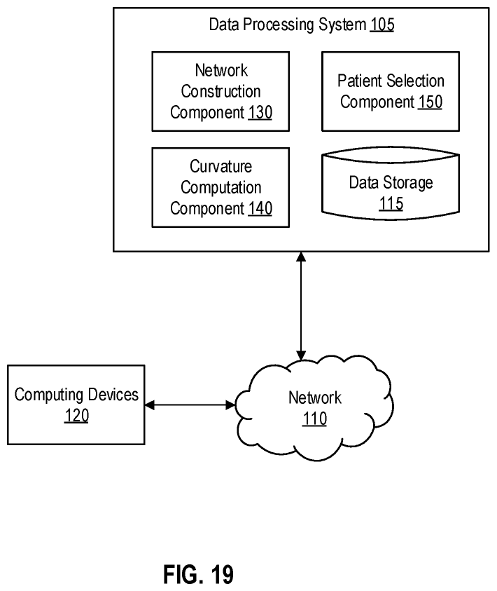

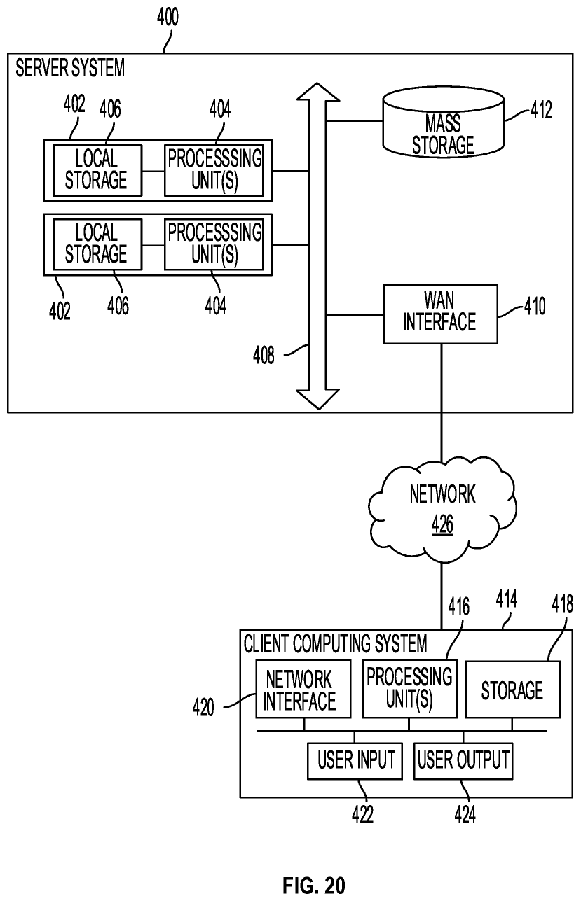

Referring now to FIG. 19, depicted is an example system for performing network curvature detection and corresponding patient selection, in accordance with one or more implementations. In brief overview, the system can include at least one data processing system 105, at least one network 110 (which may be the same as, or a part of, network 426 described herein below in conjunction with FIG. 20), and at least one computing device 120. The data processing system 105 can include at least one network construction component 130, at least one curvature computation component 140, and at least one patient selection component 150. In some implementations, the data processing system 105 can include data storage 115, and in some implementations, the data storage 115 can be external to the data processing system 105. For example, when the data storage 115 is external to the data processing system 105, the data processing system 105 (or the components thereof) can communicate with the data storage 115 via the network 110. In some implementations, the data processing system 105 can implement or perform any of the functionalities and operations discussed herein.

Each of the components (e.g., the data processing system 105 (including its components), the network 110, the computing device 120, etc.) of the system 100 can be implemented using the hardware components or a combination of software with the hardware components of a computing system (e.g., server system 400, client computing system 414, any other computing system described herein, etc.) detailed herein in conjunction with FIG. 20. Each of the components of the data processing system 105 can perform the functionalities detailed herein.

In further detail, the data processing system 105 can include at least one processor and a memory (e.g., a processing circuit). The memory can store processor-executable instructions that, when executed by processor, cause the processor to perform one or more of the operations described herein. The processor may include a microprocessor, an application-specific integrated circuit (ASIC), a field-programmable gate array (FPGA), etc., or combinations thereof. The memory may include, but is not limited to, electronic, optical, magnetic, or any other storage or transmission device capable of providing the processor with program instructions. The memory may further include a floppy disk, CD-ROM, DVD, magnetic disk, memory chip, ASIC, FPGA, read-only memory (ROM), random-access memory (RAM), electrically erasable programmable ROM (EEPROM), erasable programmable ROM (EPROM), flash memory, optical media, or any other suitable memory from which the processor can read instructions. The instructions may include code from any suitable computer programming language. The data processing system 105 may include one or more computing devices or servers that can perform various functions as described herein. The data processing system 105 can include any or all of the components and perform any or all of the functions of the server system 400 or the client computing system 414 described herein below in conjunction with FIG. 20.

The network 110 can include computer networks such as the Internet, local, wide, metro or other area networks, intranets, satellite networks, other computer networks such as voice or data mobile phone communication networks, and combinations thereof. In some implementations, the network 110 can be, be a part of, or include one or more aspects of the network 426 described in connection with FIG. 20. The data processing system 105 of the system 100 can communicate via the network 110, for instance with at least one computing device 120. The network 110 can be any form of computer network that can relay information between the data processing system 105, the computing device 120, and in some implementations one or more external or third-party computing devices, such as web servers, among others. In some implementations, the network 110 can include the Internet and/or other types of data networks, such as a local area network (LAN), a wide area network (WAN), a cellular network, a satellite network, or other types of data networks.

The network 110 can also include any number of computing devices (e.g., computers, servers, routers, network switches, etc.) that are configured to receive and/or transmit data within the network 110. The network 110 can further include any number of hardwired and/or wireless connections. Any or all of the computing devices described herein (e.g., the data processing system 105, the computing device 120, the server system 400, the client computing device 414, etc.) can communicate wirelessly (e.g., via WiFi, cellular, radio, etc.) with a transceiver that is hardwired (e.g., via a fiber optic cable, a CAT5 cable, etc.) to other computing devices in the network 110. Any or all of the computing devices described herein (e.g., the data processing system 105, the computing device 120, the server system 400, the client computing device 414, etc.) can also communicate wirelessly with the computing devices of the network 110 via a proxy device (e.g., a router, network switch, or gateway).

The data storage 115 can be a database configured to store and/or maintain any of the information described herein. The data storage 115 can maintain one or more data structures, which may contain, index, or otherwise store each of the values, pluralities, sets, variables, vectors, thresholds, or any generated or determined information described herein. The data storage 115 can be accessed using one or more memory addresses, index values, or identifiers of any item, structure, or region maintained in the data storage 115. The data storage 115 can be accessed by the components of the data processing system 105, or any other computing device described herein, via the network 110. In some implementations, the data storage 115 can be internal to the data processing system 105. In some implementations, the data storage 115 can be external to the data processing system 105, and may be accessed via the network 110. The data storage 115 can be distributed across many different computer systems or storage elements, and may be accessed via the network 110 or a suitable computer bus interface.

The data processing system 105 can store, in one or more regions of the memory of the data processing system 105, or in the data storage 115, the results of any or all computations, determinations, selections, identifications, generations, constructions, or calculations in one or more data structures indexed or identified with appropriate values. Any or all values stored in the data storage 115 may be accessed by any computing device described herein, such as the data processing system 105, to perform any of the functionalities or functions described herein.

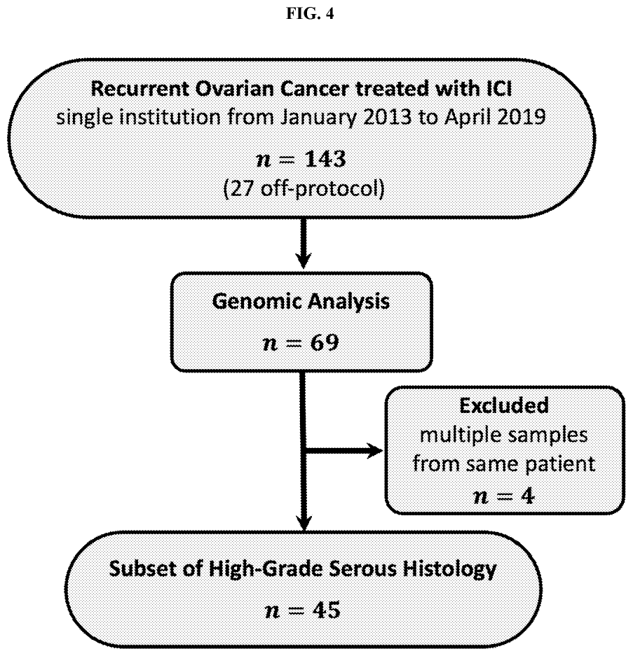

The computing device 120 can include at least one processor and a memory, e.g., a processing circuit. The memory can store processor-executable instructions that, when executed by processor, cause the processor to perform one or more of the operations described herein. The processor may include a microprocessor, an ASIC, an FPGA, etc., or combinations thereof. The memory may include, but is not limited to, electronic, optical, magnetic, or any other storage or transmission device capable of providing the processor with program instructions. The memory may further include a floppy disk, CD-ROM, DVD, magnetic disk, memory chip, ASIC, FPGA, ROM, RAM, EEPROM, EPROM, flash memory, optical media, or any other suitable memory from which the processor can read instructions. The instructions may include code from any suitable computer programming language. The computing device 120 can include one or more computing devices or servers that can perform various functions as described herein. The computing device 120 can include any or all of the components and perform any or all of the functions of the server system 400 or the client computing system 414 described herein below in conjunction with FIG. 4.

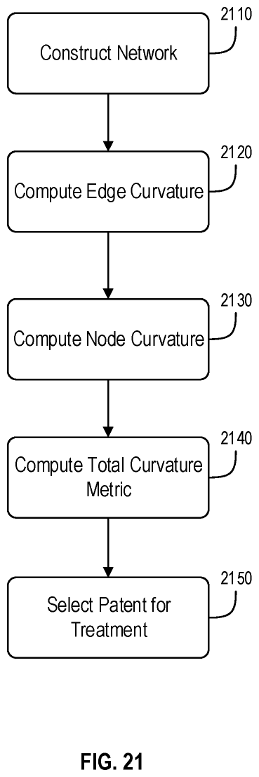

The network construction component 130 can construct a genomic network that includes a plurality of nodes and edges representing a plurality of gene pathways. FIG. 21 depicts a corresponding method that includes a similar operation 2110 for constructing a network. The network construction component 130 may construct the network based on one or more evidenced-based interactions and/or inputs provided by a protein-protein ineractome database (e.g., the Human Reference Protein Database (HPRD), the Human Reference INteractome (HuRI), Search Tool for the Retrieval of Interacting Genes/Proteins (STRING) etc.). Such genomic networks have a topology (e.g., a connectivity structure) and a geometry (including a curvature) that gives a measure of their functional robustness. Each node of the network is a gene that is a component of a gene pathway and each node is associated with a copy number alteration (CNA) metric. The CNA metric for each node is based on genomic sequence data corresponding to at least one tumor of the patient. The genomic sequence data may be obtained by sequencing circulating tumor DNA (ctDNA) or sequencing DNA from a biopsied tumor isolated from the patient. In some embodiments, the genomic sequence data is obtained via whole exome sequencing or targeted exome sequencing.

Additionally or alternatively, in some embodiments of the methods described herein, the plurality of nodes of the network comprises ABL1, ACTB, ACTG1, ACTN1, ACVR1, ADAM15, AKT1, AKT2, APP, AR, ARRB2, ATM, ATXN1, AXIN1, BCAR1, BCL2, BCL2L1, BRCA1, BTK, C14orfl, CASP8, CCND1, CCND3, CCNE1, CD247, CDC42, CDK4, CDK5, CDKN1A, CDKN1B, CHD3, COIL, COPS5, COPS6, CREBBP, CRK, CRMP1, CSNK2A2, CTNNB1, DAXX, DLG4, DNM2, DVL2, EGFR, EIF2AK2, EP300, ESR1, EWSR1, EZR, FASLG, FEZ1, FGFR1, FN1, FOS, FXR2, GFI1B, GNAI1, GRB2, GSK3B, HCK, HDAC3, HGS, HIPK2, HRAS, HSF1, HSP90AA1, HTT, IGF1R, INSR, ITGB1, JAK1, JAK2, JAK3, JUN, LYN, MAGEA11, MAP2K1, MAP3K7, MAPK1, MAPK14, MAPK8, MCM2, MCM7, MDF1, MDM2, MLLT4, MUC1, MYC, MYOC, NCOA2, NCOR1, NEDD4, NFKBIA, NINL, NOTCH1, NR3C1, NTRK1, PAK1, PARP1, PCNA, PDPK1, PIAS1, PIK3CA, PIK3R1, PIK3R2, PLCG1, PLG, PML, POLR2A, POU2F1, PPP2R5A, PRKCA, PRKCB, PRKCD, PRKCE, PRKCG, PRNP, PRSS23, PSEN1, PTK2, RAC1, RAD51, RAF1, RANBP9, RARA, RASA1, RB1, RBL1, RBPMS, RGS2, RHOA, RPA1, RUNX2, RXRG, SAT1, SH3KBP1, SHC1, SLC9A3R1, SMAD2, SMAD3, SMAD4, SMAD7, SMARCA4, SMURF1, SNAPIN, SRC, STAT1, SUMO1, SUMO2, SUMO4, SUV39H1, SVIL, SYK, SYN1, TGFBR1, TGFBR2, TGM2, TLE1, TP53, TRAF6, TRIP13, UBB, UPF1, UTP14A, VIM, WAS, XPO1, XRCC6, YAP1, YWHAE, and YWHAQ. In certain embodiments, the plurality of nodes of the network comprises ACTB, ACVR1, ADAM15, AKT1, APP, AR, ARRB2, ATXN1, AXIN1, BCL2, BRCA1, BTK, CASP8, CD247, CDC42, CDK5, CDKN1A, CHD3, COIL, COPS6, CREBBP, CRK, CRMP1, CSNK2A2, CTNNB1, DLG4, DVL2, EGFR, EIF2AK2, EP300, ESR1, EWSR1, FASLG, FGFR1, FN1, FXR2, GNAI1, GRB2, GSK3B, HDAC3, HGS, HIPK2, HRAS, HSF1, HSP90AA1, HTT, JAK1, JUN, LYN, MAGEAI1, MAPK1, MAPK14, MDM2, MUC1, MYC, MYOC, NCOR1, NR3C1, NTRK1, PAK1, PARP1, PCNA, PDPK1, PIK3R1, PIK3R2, PLCG1, POLR2A, POU2F1, PPP2R5A, PRKCA, PRKCD, PRKCE, PTK2, RAC1, RAF1, RANBP9, RASA1, RB1, RHOA, RPA1, SHC1, SMAD2, SMAD3, SMAD4, SMAD7, SMARCA4, SMURF1, SNAPIN, SRC, STAT1, SUMO1, SUMO4, TGFBR1, TP53, UBB, VIM, XPO1, XRCC6, YWHAE, and YWHAQ.

The curvature computation component 140 can analyze the genomic network and determine therefrom one or more curvature metrics based on computations involving characteristics of the network. For example, as indicated in operation 2120 of FIG. 21, the curvature computation component 140 computes an edge curvature for each of the plurality of edges, for example, by using an Ollivier-Ricci curvature equation such as Equation 2 described below. This edge curvature determination may further be based on a weighted hop distance between two nodes associated with a respective edge as discussed for example throughout this description, and the weighted hop distance determined using node weights associated with the CNA metrics for the two nodes associated with the respective edge. The weighted hop distance is indicative of a likelihood of interaction between the genes associated with the two nodes associated with the respective edge. Examples equations for implementing such a weighted hop distance are described below with respect to Equations 6-8.

The curvature computation component 140 further computes a node curvature for for each of the plurality of nodes of the network as indicated for example in operation 2130 of FIG. 21. An example of an equation for such a node curvature is provided below in Equation 9. This computation may be based on the edge curvatures for the edges of the network incident to each of the nodes for which the node curvature is being determined. The node curvature may include a scalar curvature for a corresponding gene determined from a weighted sum of curvatures on all edges incident to the corresponding gene.

The curvature computation component 140 also computes a total curvature metric for the network by determining a net node curvature over a plurality of the nodes of the network as indicated for example in operation 2140 of FIG. 21. In one example embodiment, the net node curvature is determined over all nodes of the network to compute the total curvature metric using, for example, Equation 12 as described below. The total curvature metric may include a net scalar curvature summed over all nodes of the network.

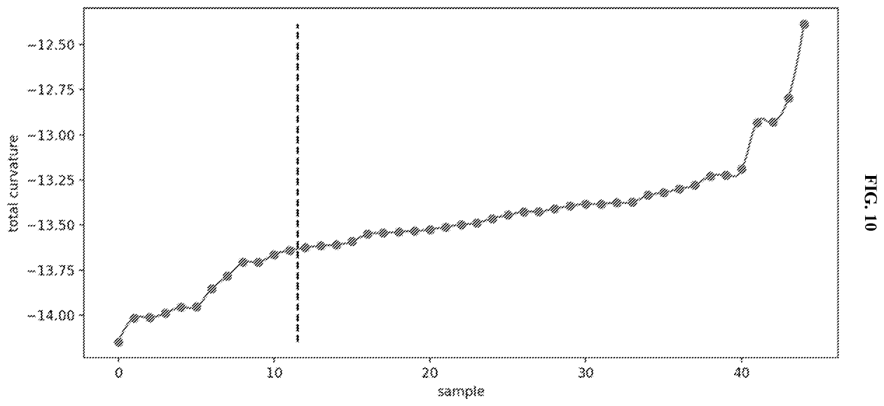

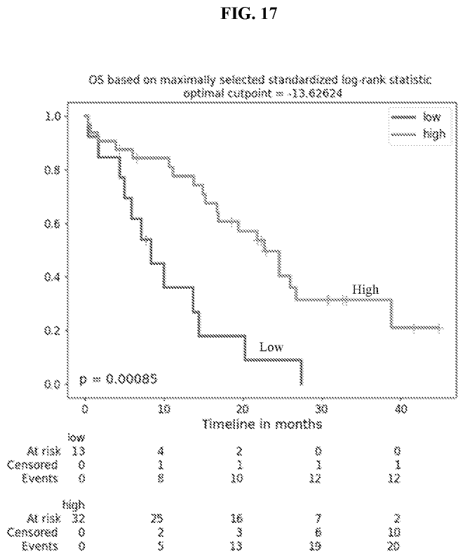

The patient selection component 150 selects a patient for treatment with an immune checkpoint inhibitor therapy when the patient exhibits a total curvature metric that satisfies a threshold as indicated for example in operation 2150 of FIG. 21. In some embodiments, the threshold corresponds to total curvature metrics observed in ovarian cancer patients that are responsive to immune checkpoint inhibitor (ICI) therapy. Responsiveness to ICI therapy may be determined via iRECIST criteria or tumor shrinkage. In an embodiment, the threshold may correspond to a location or value associated with where a curve fitted to the total curvature metric values begins to increment and is approximately linear. Such an example location or value is indicated by the vertical dashed line at approximately sample 11 in FIG. 10. Accordingly, patients associated with a total curvature metric that satisfies (or exceeds) the value associated with the location where the curve fitted with where the total curvature metric values begin to linearly increment are selected for treatment. In another embodiment, the threshold may be selected using a maximally selected log-rank statistic as demonstrated in FIG. 17 such that patients having total curvature metrics that satisfy such a statistic are selected.

Additionally or alternatively, in some embodiments of the methods disclosed herein, the immune checkpoint inhibitor therapy comprises one or more of an anti-PD-1 antibody, an anti-PD-L1 antibody, an anti-PD-L2 antibody, an anti-CTLA-4 antibody, an anti-TIM3 antibody, an anti-TIGIT antibody, an anti-VISTA antibody, an anti-B7-H3 antibody, an anti-BTLA antibody, an anti-CD73 antibody, or an anti-LAG-3 antibody. In certain embodiments, the immune checkpoint inhibitor therapy comprises one or more of pembrolizumab, nivolumab, cemiplimab, atezolizumab, avelumab, durvalumab, ipilimumab, tremelimumab, ticlimumab, JTX-4014, Spartalizumab (PDR001), Camrelizumab (SHR1210), Sintilimab (IB1I308), Tislelizumab (BGB-A317), Toripalimab (JS 001), Dostarlimab (TSR-042, WBP-285), INCMGA00012 (MGA012), AMP-224, AMP-514, KN035, CK-301, AUNP12, CA-170, or BMS-986189.

### EXAMPLES

The present technology is further illustrated by the following Examples, which should not be construed as limiting in any way.

### Example 1: Materials and Methods

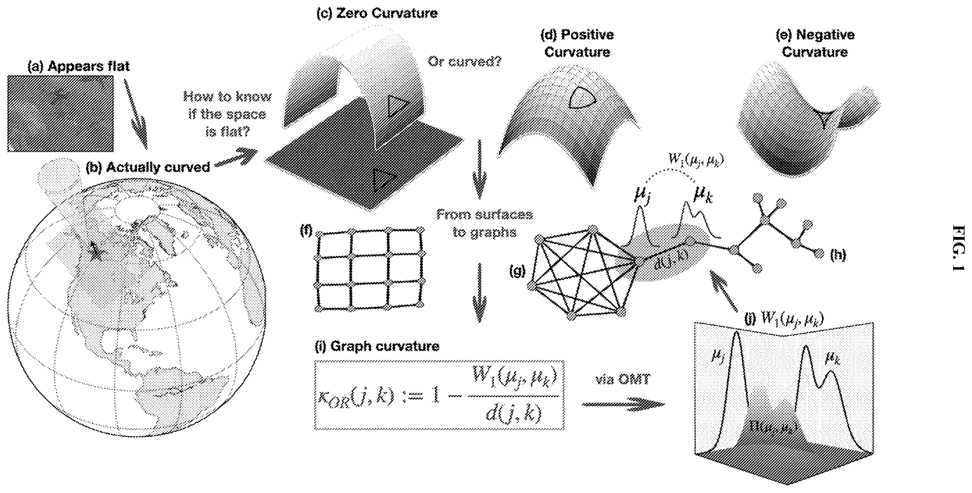

Curvature background: Perhaps the most intuitive notion of curvature is that of Gaussian curvature on a surface as shown in FIG. 1 (summarizing the relationship between the continuous and discrete notions of curvature). The curvature proposed by Ollivier is the discrete analogue of Gaussian curvature on a surface, and more generally, of Ricci curvature on higher dimensional objects. Application of this generalized, abstracted notion of curvature is proposed for studying cancer networks, as elucidated below. The key point is that the notion of curvature employed in this disclosure is intrinsic to the given geometric object. For networks defined by graphs, one looks at such an intrinsically defined quantity to inform on its (functional) structure.

In the classical case, the Gaussian curvature of a surface is independent of how the surface is embedded in 3-dimensional space. Thus, rather than look at the surface as it is embedded in 3-dimensional space from the perspective of an outsider, the key is to treat the surface as the space itself. With this approach, it can be determined if the space is curved through the use of geodesics, the curves of (locally) shortest length between two points. (Geodesics generalize straight lines in Euclidean space.) One way to tell if the space is curved is to sum up the interior angles of a geodesic triangle. Geodesic triangles on a surface with positive (resp., negative) Gaussian curvature are fat (resp., skinny) compared to the triangle in Euclidean space. Loosely speaking, curvature can be inferred by the local behavior of geodesics—geodesics converge in regions of positive curvature and diverge in regions of negative curvature. On Riemannian manifolds, Ricci curvature is intimately related to the spread of geodesics emanating from the same point.

While there are many ways to characterize the local behavior of Ricci curvature, Ollivier's characterization is most relevant for the purposes of this disclosure: namely that in regions of positive (resp., negative) Ricci curvature, geodesic balls (on average) are closer (resp., farther) than their centers. (A “geodesic ball” of radius E centered at a given point p is defined as the image under the exponential map of the ball of radius E on the tangent space at p). This is in contrast to Euclidean space where the distances between geodesic balls and their centers are the same. Ollivier's characterization generalizes this notion of Ricci curvature applicable to graphs by replacing the geodesic balls with probability measures μⱼ. In the Euclidean case, one may think of this as replacing points (delta functions) by small Gaussian balls (“fuzzified points”). The transportation distance between measures μⱼ and μₖ, prescribed by the Wasserstein distance W₁, is used in lieu of the average distance between geodesic balls. The Wasserstein distance accounts for the geometry of the space and the distance between distributions associated with two nodes is related to the overlap of their neighborhoods. The rigorous mathematical details will be given now.

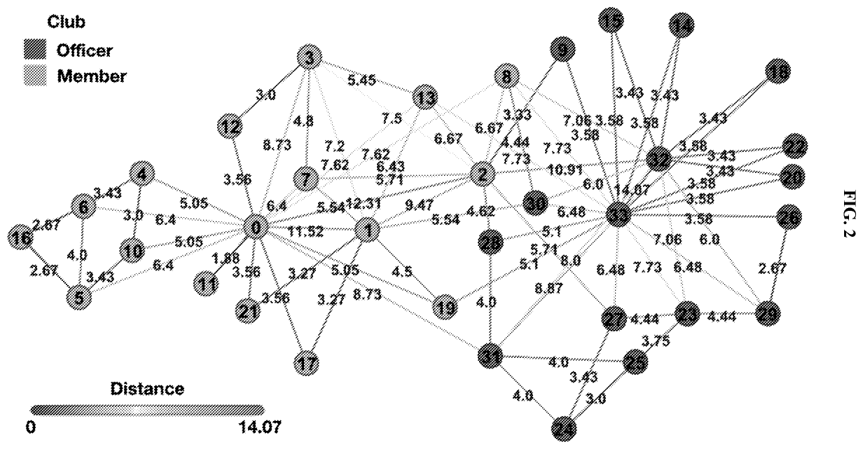

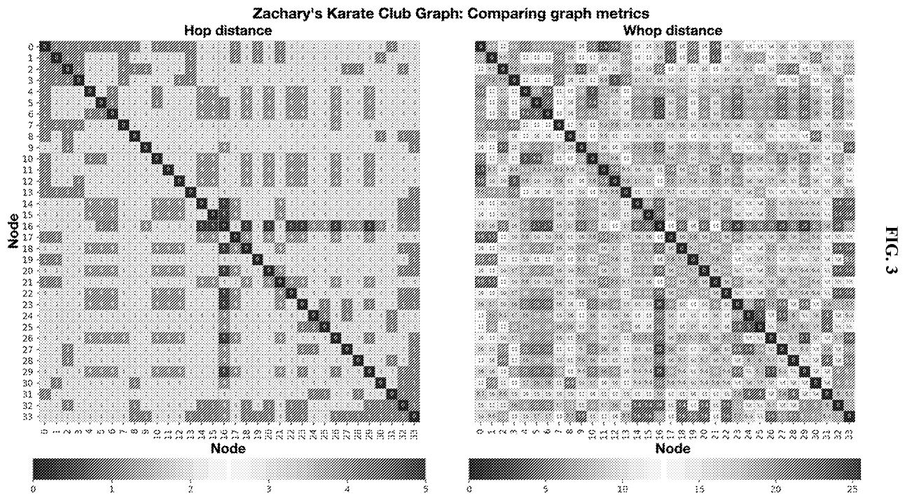

Weighted hop distance. Using Zachary's Karate Club graph as an example, the resulting weighted hop (whop) distance for all edges is shown in FIG. 2. A more detailed comparison between the hop and whop distances, illustrated by heat maps of the corresponding distance matrices of all node pairs in the network, is shown in FIG. 3.

Wasserstein distance: The Wasserstein distance is a particular instance of the optimal mass transport (OMT) problem. It is a natural candidate for comparing probability measures because it accounts for both the shape of the distributions (i.e., weighted values) and the distance on the underlying space. The OMT problem seeks the optimal way to redistribute mass with minimal transportation cost. Consider the following discrete formulation; since the theory will be applied to weighted graphs, this will be sufficient.

Accordingly, let X denote a metric measure space equipped with distance d(·,·). Given two (discrete) probability measures μ₀ and μ₁ on X, the Wasserstein distance W₁ between μ₀ and μ₁ is defined as

$${W}_{1} ( {μ}_{0} ; {μ}_{1} ) := \text{?} {\sum }_{x , y} {π}_{xy} dx , y , \qquad ( 1 )$$

$$\text{?} \text{indicates text missing or illegible when filed}$$

where Π(μ₀, μ₁) is the set of joint probabilities on X×X with marginals μ₀ and μ₁. Here, π_{xy} may be interpreted as the amount of mass moved from x toy and the cost of transporting a unit of mass is taken to be the distance travelled (i.e., d). Thus, the Wasserstein distance (1) gives the minimal net cost of transporting mass distributed by μ₀ to match the distribution of μ₁. The OMT problem therefore seeks the optimal transference plan π∈Π(μ₀, μ₁) found to be the infimal argument for which the Wasserstein distance is realized.

Curvature: The interplay between Ollivier-Ricci curvature, network entropy and functional robustness is linked by optimal mass transport (OMT), and is rich in theory.

Based on the work of von Renesse and Sturm, Ollivier extended the notion of Ricci curvature, defined on a Riemannian manifold, to discrete metric measure spaces. Specifically, let X be a metric measure space equipped with a distance d such that for each x∈X one is given a probability measure μₓ. The probability measure μₓ can be thought of as fuzzifying or blurring the point x. For two points x, y∈X, Ollivier-Ricci curvature is defined as

$${κ}_{OR} ( x , y ) := 1 \frac{{W}_{1} ( {μ}_{x} , {μ}_{y} )}{d ( x , y )} , \qquad ( 2 )$$

where W₁ is the Wasserstein distance.

Curvature on graphs: The metric measure space is taken to be a weighted graph G=(V,E) with nodes (vertices) V and edges E. G is assumed to be a simple, connected and undirected graph. Instead of points x in a metric space, consider nodes xⱼ∈V, denoted simply by its subscript j. In this work, the graph is constructed as follows. Each node j∈V represents a gene; hereafter node and gene are used interchangeably. Edges e=(j, k)∈E define known interactions between genes (nodes) at the protein level (here given by HPRD) and j~k denotes that k is a neighbor of j. Copy number (CN) values are incorporated as nodal weights, denoted wⱼ. Note that for j∈V, wⱼ=(CN)ⱼ+1; the affine translation is used to ensure all weights are positive.

The weighted graph was treated as a Markov chain. In this context, the probability measure μⱼ attached to node j∈V can be thought of as the probability of a 1-step random walk starting from node j. The 1-step transition probability pⱼₖ of going from j to k is expressed by the principle of mass action. According to this principle, if there is a known connection between gene j and gene k (i.e., (j, k)∈E), then the probability that they interact is proportional to the product of their CN values:

$${p}_{j k} ∝ {w}_{j} {w}_{k} . \qquad ( 3 )$$

Normalizing the mass action over all possible edges to ensure that pⱼₖ is a probability, i.e., Σ_{j~k}Pⱼₖ=1, the transition probabilities pⱼₖ of the stochastic matrix P=[pᵢⱼ] associated with the Markov chain is defined as follows:

$${p}_{j} k = \{ \begin{matrix}\frac{{w}_{k}}{{\sum }_{j \sim k} {w}_{k}} , & if j \sim k \\ 0 , & otherwise\end{matrix} . \qquad ( 4 )$$

Accordingly, for each gene j, a probability measure μⱼ defined on the node set V with n associated nodes is

$${μ}_{j} = [ {p}_{j 1} , {p}_{j 2} , {p}_{jn} ] , j = 1 , … , n . \qquad ( 5 )$$

Alternatively, μⱼ can be thought of as fuzzifying the node j over its 1-step neighborhood.

Graph distance: The points (x) and measures (μₓ) needed to compute OR curvature in Eq. (2) on a graph have been determined. All that remains is the distance d(x,y). In lieu of the commonly used hop distance, i.e., the distance between two nodes j, k∈V that is defined as the shortest path length over all paths connecting j and k, the corresponding graph distance dⱼₖ to be the weighted hop distance (whop) is utilized.

More precisely, for fixed nodes j and k, let P^{jk} denote a path connecting them. Let {wᵢ^{jk} . . . wₙ^{jk}} be the set of all the associated edge weights. Then the equation is set as

$$ℓ ( {P}^{jk} ) := {\sum }_{i = 1}^{n} \frac{1}{{w}_{i}^{jk}} . \qquad ( 6 )$$

Denoting by,

$$𝒫 := \{ {P}_{1}^{jk} , … , {P}_{m}^{jk} \} ,$$

the set of all possible paths connecting j and k, the weighted hop distance (whop) between j and k is defined to be:

$${d}_{jk} := {min}_{1 \leq u \leq m} ℓ ( {P}_{u}^{jk} ) . \qquad ( 7 )$$

Note that the edge weights wᵤᵥ for all edges e=(u, v)∈E are constructed as

$${w}_{uv} := \frac{{p}_{uv} + {p}_{uv}}{2} \qquad ( 8 )$$

This formulation was chosen so the distance between two nodes is inversely related to the probability of their interaction. Thus, the higher (resp., lower) the probability of two nodes interacting, the smaller (resp., larger) the distance between them should be. The average is taken merely so the distance is symmetric, i.e., dⱼₖ=dₖⱼ. See FIGS. 2 and 3 for an explicit example of the weighted hop distance on a simple network.

Edge curvature: With the choice of graph distance in Eq. (7), the OR curvature in Eq. (2) can now be computed between any two nodes in the graph. Due to the large nature of the graphs of interest, the curvature computation is constrained to edges. Notice, from the curvature definition in Eq. (2), the ratio

$$\frac{{W}_{1} ( {μ}_{j} , {μ}_{k} )}{{d}_{jk}}$$

relates the transport cost of moving the distribution (i.e., fuzzy ball) associated with j to k to the ground distance. Informally, the more the neighborhoods of two nodes overlap, the lower the transportation cost between them and thus the higher the curvature associated with the edge. As such, curvature informs on the local functional relationship between neighborhoods.

Scalar and total curvature on graphs: In order to obtain a node-level measure of curvature, a contraction of the edge curvatures is considered, analogous to scalar curvature defined on points of a manifold in Riemannian geometry. Motivated by the notion of signaling entropy rate in information theory, the (nodal) scalar curvature of gene j to be the weighted sum of the curvatures on all edges incident to j is defined as:

$${κ}_{j} := {π}_{j} {\sum }_{j \sim k} {κ}_{OR} ( j , k ) \qquad ( 9 )$$

where the weight πⱼ is the j^{th} component of the stationary distribution T associated with the Markov chain P [39]:

$$π = π P , {\sum }_{j} {π}_{j} = 1. \qquad ( 10 )$$

The stationary distribution in this setting (connected graph) is also the limiting distribution of the Markov chain, known as the stationary or equilibrium distribution. Thus, the quantity πⱼ describes the relative importance of node j with respect to all other nodes. The nodal curvature is then scaled by its component in the stationary distribution in order to correct for nodal bias. Furthermore, the stationary distribution has a closed form that may be easily computed as follows:

$${π}_{j} = \frac{1}{Z} {w}_{j} {\sum }_{j \sim k} {w}_{k} \qquad ( 11 )$$

where Z is the normalization factor. Of note, unweighted and alternative weightings have been proposed. Lastly, the total curvature κ_{G} of a network is defined to be the net scalar curvature, summed over all nodes in the graph

$${κ}_{G} := {\sum }_{j} {κ}_{j} . \qquad ( 12 )$$

Curvature and robustness: One of the main motivations for using curvature to study networks in general, and biological networks in particular, is its theoretical connection to network robustness. Given its importance, there is an argument which also gives a justification for using Ollivier-Ricci curvature.

Sturm, Lott and Villani related a lower bound on the Ricci curvature of a smooth Riemannian manifold to the entropy of densities along a constant-speed geodesic with the use of the Wasserstein distance. This laid the groundwork for the connection between curvature, entropy, and the Wasserstein metric, and led to the remarkable observation that changes in Ricci curvature R_{ic} are positively correlated with changes in (Boltzmann) entropy ΔS:

$$Δ {κ}_{Ric} \times Δ S \geq 0. \qquad ( 13 )$$

The positive correlation between changes in curvature Δκ_{Ric} and changes in robustness ΔR:

$$Δ {κ}_{Ric} \times Δ R \qquad ( 14 )$$

is realized by Eq. (13) and the fluctuation theorem from large deviations theory indicates that changes in entropy are positively correlated with changes in robustness ΔR

$$Δ S \times Δ R . \qquad ( 15 )$$

Here, robustness refers to the ability of a system to recover or maintain its ability to function after it is perturbed in some way (e.g., stress signal). The Ollivier-Ricci curvature on networks is directly derived from the Lott-Sturm-Villani relationship, and thus was chosen over other possible discrete models.

Curvature's intimate connection to robustness makes it a particularly attractive method for analyzing key nodes and interactions in large, complex PPI networks. This connection is linked by entropy as shown in Eqs. (13, 15), bridging this geometric analysis to an interesting perspective on the relationship between the topological and functional properties of the weighted network. With this notion of the change in curvature as a proxy for the more qualitative notion of functional robustness, genes are ranked according to the change in curvature with respect to the topology and between sub-groups identified; see following Examples.

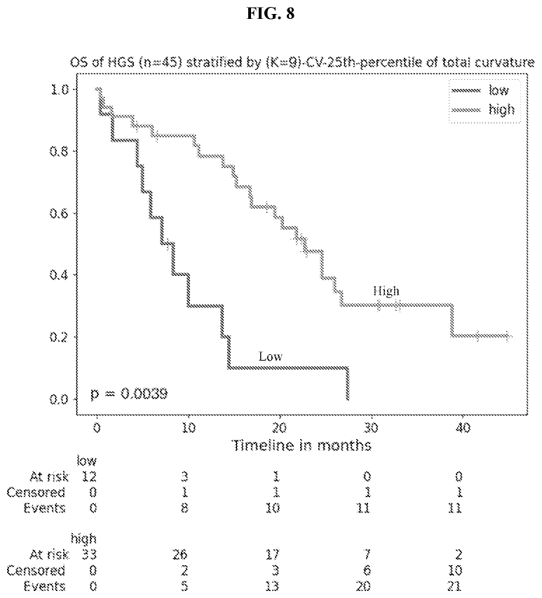

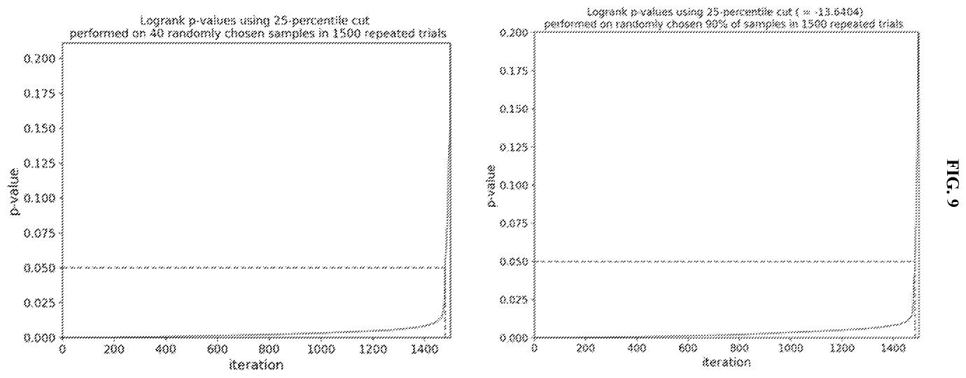

Validation of survival analysis in HGSOC. Curvature is used as a relative measure of network response to immunotherapy in HGSOC for predicting survival. An independent data set for HGSOC patients treated with immunotherapy was not available for external validation and the sample size was too small to separate into training and validation sets. We therefore performed three tests for internal validation. K-fold cross-validation (K-CV) (FIG. 8) and bootstrap validation (FIG. 9) were performed to test the validation. In addition, to test if the significant difference in survival found between the high and low curvature groups would likely be observed regardless of the initial gene level data, the CN data was randomly permuted and reassigned amongst the genes. The curvature pipeline was subsequently reperformed with the randomized node weightings and the survival between the two groups defined by low and high curvature according to the 25th percentile of the total curvature value was reassessed in the same manner using the log-rank test. This random process was repeated 500 times with zero out of the 500 trials resulting in a log-rank test statistic greater than the unpermuted sample based test statistic, suggesting that the null hypothesis may fairly be rejected and that total curvature is statistically likely to be picking up on real signals of functional robustness providing a relative measure of overall survival in response to immunotherapy.

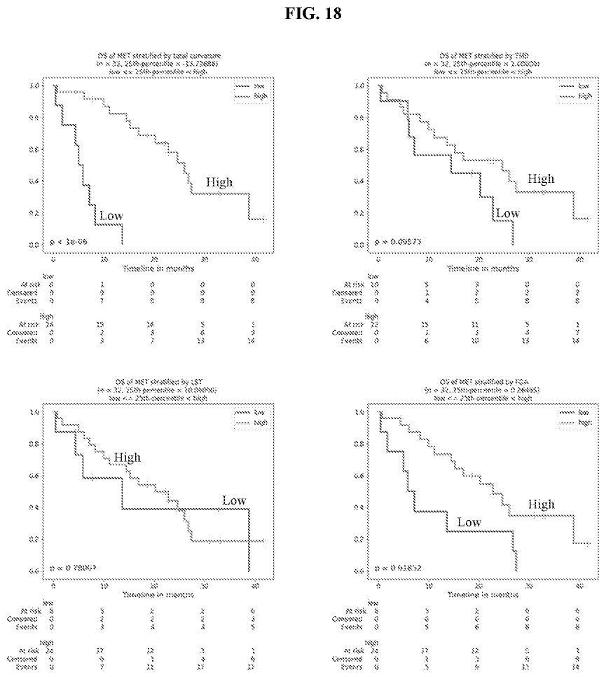

Survival analysis on metastasis cohort. Kaplan-Meier survival analysis as performed in FIG. 1 was repeated on the metastasis cohort (n=32). The corresponding survival curves and log-rank p-values are shown in FIG. 18.

Data description and processing: In this section, the data description and processing used in the HGSOC analysis is outlined.

First of all, TMB was calculated by dividing the number of non-synonymous mutations by the total size of the capture panel in megabases. Secondly, based on the CNAs by FACETS, FGA was defined as the cumulative length of segments with log 2 or linear CNA value larger than 0.2 divided by the cumulative length of all segments measured. LST scores, defined as a chromosomal breakpoint resulting in allelic imbalance between adjacent regions of at least 10 Mb, were determined, and a cut-off 15 was employed for LST-high cases.

Next, regarding the data characteristics, DNA gene CNA data from a subset of 69 women with recurrent OC who received immunotherapy from a previously published series was utilized. The subtypes of ovarian cancer are in fact quite different diseases, originating in different cell types and being caused by distinct mutations with diverse outcomes, and should therefore be analyzed separately. Accordingly, the re-analysis is restricted to a subset of samples (n=49) with HGSOC, which is the most common and lethal subtype. Four HGSOC patients had two samples, and the replicate samples were removed from the analysis. This resulted in a total of 45 tumor samples, 32 of which were metastases and 13 represented primary (adnexal) tumors, with 22 and 10 deaths in each group, respectively, at the time the study group was analyzed. This forms a homogeneous group of cancers (FIG. 4 and Table 1). Tumor and normal samples from the 45 patients were profiled utilizing the FDA-cleared Memorial Sloan Kettering Integrated Mutation Profiling of Actionable Cancer Targets (MSK-IMPACT) sequencing assay, their mean age was 58 years, and mean TMB was 5.9. Patient selection and clinical characteristics are displayed in FIG. 4 and in Tables 1 and 2.

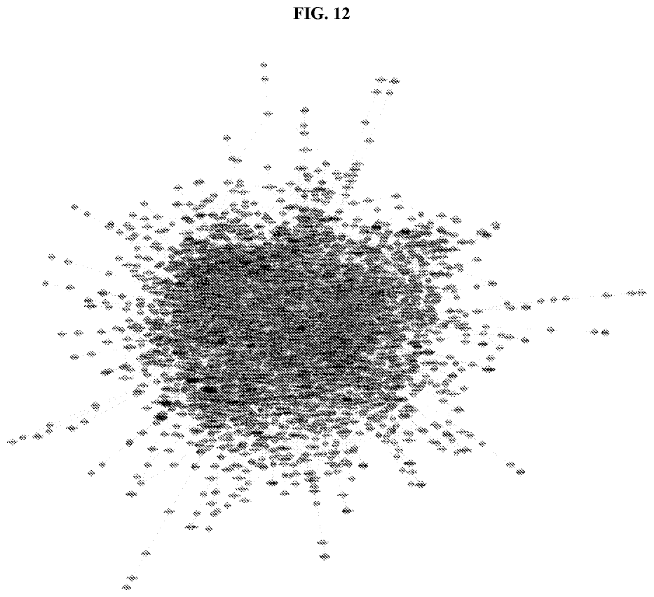

CN segments were mapped to individual genes according to GRCh37 and for each sample, each gene was assigned the maximum CN value of all segments that mapped to it. After removing all genes with missing data and all genes not in the HPRD network, the set of genes comprising the largest connected network were extracted (FIG. 12). This resulted in a CNA data matrix of size 3,489 (genes)×45 (samples).

The network topology was constructed as follows. Edges between genes were defined by the PPI obtained from HPRD. The network topology was then taken to be the largest connected component in the HPRD network restricted to the set of genes in the data set. This resulted in a network with 9,710 edges and 3,489 nodes with an average degree of 5.57. The rationale is that the established interactions between gene products serve as a viable proxy for the functional connectivity at the gene level.

Subject specific networks were created by assigning nodal weights wⱼ prescribed by the CN value. Specifically, the CN data took on discrete integer values in the range [0, 38]. In order to ensure all weights were positive, the translation wⱼ=xⱼ+1 where xⱼ is the CN value for gene j was employed. For each subject, Markov chains were computed as defined in Eq. (4) followed by the associated stationary distribution in Eq. (11). Next, Ollivier-Ricci curvature using Eq. (2) was computed on each edge in the fixed network, scalar curvature defined in Eq. (9) was subsequently computed for each node and lastly, total curvature using Eq. (12) was computed for the network. A critical aspect of the curvature analysis is that it provides a relative quantity and it is the change in curvature that is of interest, indicative of changes in the network's capacity for communication. Thus, it is expected that patients whose samples have a lower total curvature (i.e., a relative net decrease in capacity) would be associated with a poorer prognosis than those with higher total curvature values.

Tables 5-7 list the top ranked genes according to the criteria described herein. Both the greatest positive and negative curvature differences are listed. Finally, Table 8 lists (in alphabetical order) the key genes found by the curvature analysis using all the comparisons, and then in Table 9, the 100 identified candidate genes based on risk are listed alphabetically.

Sub-curvature survival analysis. The Kaplan-Meier analysis suggests that total curvature as a network measure of functional robustness is a more effective indicator of survival in HGSOC treated with ICIs than other genomic parameters. However, it was expected that not all genes in the network were necessary to predict survival. We therefore defined the sub-curvature_{g} for any subset of genes g to be the sum of scalar (node) curvatures over all genes in the given subset:

$${κ}_{g} = {\sum }_{j ∈ g} {κ}_{j} .$$

We then repeated the survival analysis replacing total curvature with sub-curvature using the following two subsets of genes: (1) the curvature identified genes consisting of the 171 unique genes top-ranked by the various network curvature criteria (Tables 5-7) listed alphabetically in Table 8 and (2) the risk identified genes consisting of the top-ranked 100 genes by the curvature risk criterion (Table 1) listed alphabetically in Table 9.

TABLE 1

| | |
|---|---|
| Variables | n = 45 |

Platinum status at ICI

| | |
|---|---|
| Sensitive | 6 (13%) |
| Resistant | 39 (87%) |

Lines of Treatment prior to ICI

| | |
|---|---|
| Median (mean) | 4 (4.6) |
| Range | 1-10 |

Type of ICI Therapy

| | |
|---|---|
| PD1/PDL1 | 25 (56%) |
| PD1/PDL1 + CTLA4 | 15 (33%) |
| PD1/PDL1 + Other | 4 (9%) |
| CTLA4 | 1 (2%) |

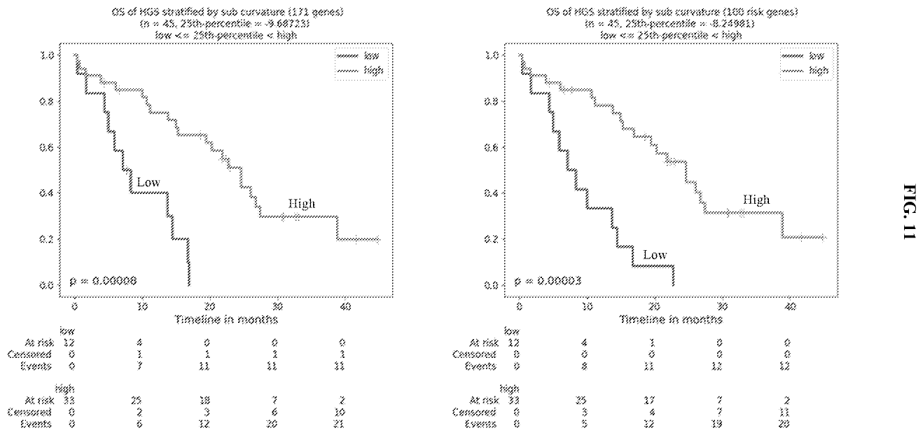

Survival curves based on the 25th-percentile of sub-curvature values for each of these subsets are shown in FIG. 11. In both cases, the p-value (171 curvature identified genes: p=0.00008; 100 risk identified genes: p=0.00003) is about 1 order of magnitude smaller as compared to the total curvature, suggesting that the curvature methodology identifies key genes pertaining to survival.

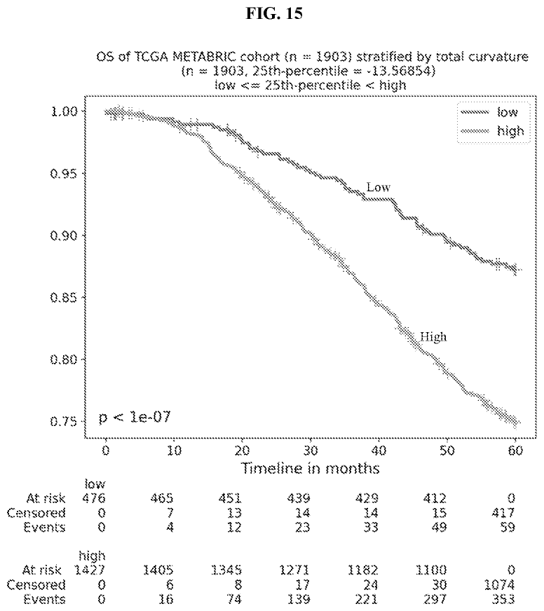

Method validation on Metabric data. For further validation, the methodology was applied to the Metabric dataset, a larger and publicly available data set with n=1903 samples. The resulting Kaplan-Meier analysis and log-rank p-value are shown in FIG. 15.

TABLE 2
HGSOC patient characteristics.

| | All patients | Low curvature | High curvature | |
|---|---|---|---|---|
| Characteristic | (n = 45) | (n = 12) | (n = 33) | p |
| Age at diagnosis (years) | | | | 0.062 |
| Mean ± SD | 58.0 ± 9.3 | 62.3 ± 7.5 | 56.4 ± 9.4 | |
| Range | 27.0-75.0 | 49.0-75.0 | 27.0-75.0 | |
| Median (IQR) | 58.0 (52.0-64.0) | 64.0 (58.8-65.5) | 55.0 (51.0-61.0) | |
| Age at start of ICI (years) | | | | 0.023 |
| Mean ± SD | 62.1 ± 8.7 | 67.1 ± 6.9 | 60.3 ± 8.7 | |
| Range | 37.0-78.0 | 55.0-78.0 | 37.0-77.0 | |
| Median (IQR) | 62.0 (56.0-69.0) | 67.0 (63.3-70.3) | 59.0 (55.0-66.0) | |
| Stage at diagnosis | | | | 0.502 |
| III | 25 | 8 | 17 | |
| IV | 20 | 4 | 16 | |
| Time from diagnosis to start of ICI (months) | | | | 0.581 |
| Mean ± SD | 50.9 ± 35.9 | 58.8 ± 43.1 | 48.1 ± 33.2 | |
| Range | 5.3-166.0 | 17.4-166.0 | 5.3-123.1 | |
| Median (IQR) | 49.4 (23.3-61.7) | 44.5 (33.4-61.8) | 49.4 (18.5-61.7) | |
| Duration of ICI (weeks) | | | | 0.807 |
| Mean ± SD | 20.2 ± 23.6 | 14.3 ± 8.6 | 22.3 ± 27.0 | |
| Range | 0.1-143.0 | 0.7-28.3 | 0.1-343.0 | |
| Median (IQR) | 12.3 (7.7-23.1) | 13.6 (7.8-20.3) | 12.1 (7.7-23.1) | |
| Overall survival (months) | | | | 0.007 |
| Mean ± SD | 16.7 ± 11.7 | 8.8 ± 7.2 | 19.6 ± 11.7 | |
| Range | 0.4-44.8 | 0.4-27.4 | 0.4-44.8 | |
| Median (IQR) | 15.3 (6.5-24.6) | 7.4 (4.9-10.9) | 20.3 (11.1-26.0) | |
| Sample type | | | | 0.010 |
| Metastasis | 32.0 | 12 | 20 | |
| Primary | 13.0 | 0 | 13 | |
| Status at last follow-up | | | | 0.134 |
| Alive | 13 | 1 | 12 | |
| Dead | 32 | 11 | 21 | |
| TMB | | | | 0.959 |
| Mean ± SD | 3.7 ± 2.3 | 3.5 ± 1.9 | 3.8 ± 2.5 | |
| Range | 1.0-9.7 | 1.1-6.7 | 1.0-9.7 | |
| Median (IQR) | 3.3 (2.0-4.4) | 2.6 (2.0-5.3) | 3.3 (2.0-4.4) | |
| FGA | | | | 0.005 |
| Mean ± SD | 0.4 ± 0.2 | 0.3 ± 0.2 | 0.5 ± 0.2 | |
| Range | 0.005-0.871 | 0.005-0.629 | 0.092-0.871 | |
| Median (IQR) | 0.4 (0.3-0.6) | (0.2 (0.1-0.4) | 0.5 (0.4-0.6) | |
| LST | | | | 0.024 |
| Mean ± SD | 25.0 ± 10.3 | 19.3 ± 9.1 | 27.1 ± 10.0 | |
| Range | 2.0-51.0 | 2.0-32.0 | 2.0-51.0 | |
| Median (IQR) | 25.0 (20.0-29.0) | 22.0 (13.5-25.8) | 27.0 (22.0-34.0) | |
| Platinum status at ICI | | | | 1.000 |
| Platinum Sensitive | 6 | 1 | 5 | |
| Platinum Resistant | 39 | 11 | 28 | |
| BRCA1/2 Status | | | | 1.000 |
| Wild-type | 35 | 9 | 26 | |
| Mutations | 10 | 3 | 7 | |
| ICI target | | | | 0.909 |
| PD-1/PD-L1 | 25 | 6 | 19 | |
| PD-1/PD-L1 + CTLA-4 | 15 | 5 | 10 | |
| PD-1/PD-L1 + other | 4 | 1 | 3 | |
| CTLA-4 | 1 | 0 | 1 | |

P values were obtained using two-sided Wilcoxon-Rank Sum test for continuous variables and Fisher-exact test for categorical variables.
SD standard deviation, IQR interquartile range.

### Example 2: Survival Analysis

The prognostic value of the total curvature κ_{G} in Eq. (12) and standard genomic parameters including TMB, the fraction of genome altered (FGA) and large-scale state transition (LST) scores (representing homologous recombination deficiency [HRD] status) were assessed with respect to the HGSOC cohort (n=45). For each parameter (κ_{G}, TMB, FGA, LST), the cohort was stratified into two groups according to the 25th percentile (low vs. high) of individual values. The cutoff was selected based on the location where the curve fitted to the sorted total curvature values starts slowly incrementing and is approximately linear (FIG. 10). An alternative cut point using maximally selected log-rank statistic was assessed as well and resulted in a comparable split (FIG. 17). However, a larger cohort is needed for further validation. The effectiveness of each parameter in terms of OS was evaluated using the Kaplan-Meier (KM) analysis.

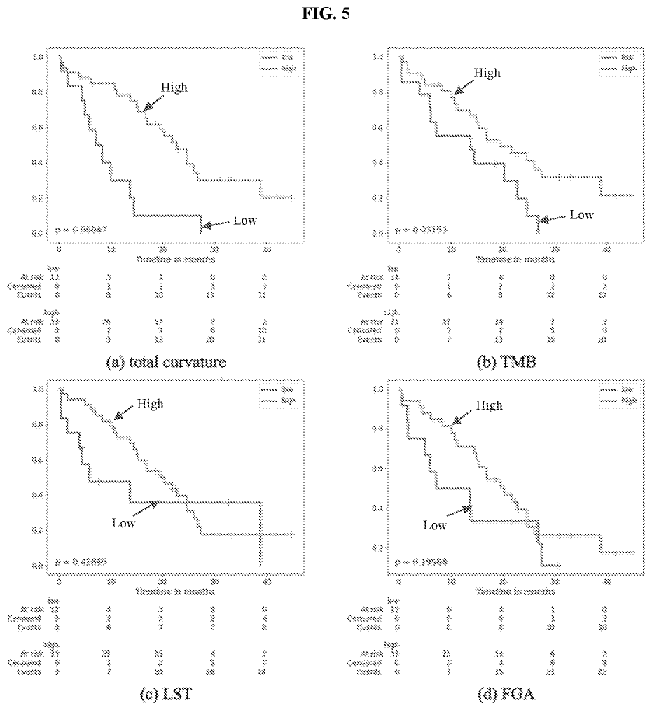

OS was defined from the start of immunotherapy treatment until either death or last follow-up. Survival curves for each parameter were plotted according to the KM estimator, shown in FIG. 5 along with the corresponding log-rank p-values (total curvature: p=0.00047; TMB: p=0.03153; LST: p=0.42865; FGA: p=0.19568). While both TMB and total curvature were found to be significant factors in predicting patient survival, the p-value for total curvature was almost 2 orders of magnitude smaller as compared to TMB, whose p-value was just marginally significant. The effective prognostic predictive power of the total curvature, particularly in comparison to the genomic parameters, is one of the major contributions of this work. See FIGS. 8 and 9 for validation and survival analysis on the metastasis subcohort (FIG. 18).

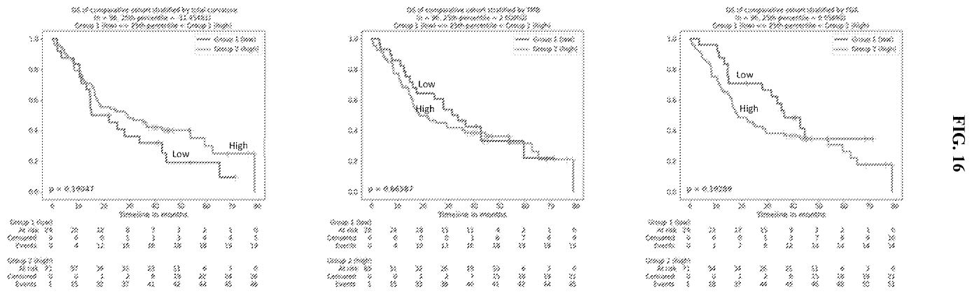

In order to assess that the prediction is not independent of receiving immunotherapy treatment, the curvature and survival analysis pipeline on IMPACT data from HGSOC samples were repeated such that it did not receive ICIs. It is interesting to note that total curvature was not predictive of survival in this setting (FIG. 16), highlighting that the findings may be immunotherapy-specific. However, it is also important to point out that OS was defined from the time of diagnosis for the analysis of this dataset, whereas in the analysis of 45 HGSOC patients treated with ICIs, OS was defined from the start date of immunotherapy, and all 45 patients had recurrent tumors with a substantial time gap between the time of first diagnosis and the start date of immunotherapy. Lastly, no statistically-significant differences were found using progression-free survival (PFS) in this cohort.

### Example 3: Functional Biomarkers

Genes that exhibit large changes in scalar curvature are identified as the genes that potentially play a key role in altering the network robustness (i.e., functional connectivity). This requires a reference for comparison, typically using data collected at a reference time (e.g., after immunotherapy treatment) or data collected from a reference sample (e.g., normal tissue). Often no such reference data are available, as was the case here where CNA data from only one time point were provided. Considering the distinction in survival curves obtained via curvature, the high and low risk groups (as previously defined by the 25th percentile of the total curvature and dichotomized into low and high curvature groups, respectively) were used for points of comparison. Genes were ranked by the difference in average scalar curvature between the low and high risk groups (Δκᵣᵢₛₖ). The change in curvature measures the relative gene implication in the stabilization (or de-stabilization) of local network robustness driving changes in feedback connectivity pertaining to survival. Since both increased and decreased functionality is of interest, the top 50 ranked genes that exhibited the largest positive (Δκᵣᵢₛₖ>0) and largest negative (Δκᵣᵢₛₖ<0) change in curvature, yielding 100 candidate genes associated with risk, are listed in Table 3 and alphabetically in Table 4.

Similarly, the top genes ranked by the difference in average scalar curvature between sub-groups based on available clinical data were investigated as an exploratory analysis. Of ancillary interest were the top ranked candidate driver genes that demonstrate functional network response to ICI and their association to survival as exhibited by disparities in network robustness measured between those who were alive or deceased at last follow-up (Δκ_{OS}; Table 5) and predominant changes in functional connectivity due to DNA level dysregulation that occurs between primary and metastatic tumors (Δκ_{PM}; Table 6).

Lastly, the network topology itself was used as a frame of reference. Treating the fixed network topology as an unweighted graph (i.e., all node weights are uniformly set to 1), the scalar curvature on this reference topology network in the same manner as detailed above was computed. This provides a measure of discordance in functional connectivity between the HGSOC network and its underlying topological structure (Δκ_{ref}; Table 7). It is interesting to note that in all of the comparisons, TP53 appeared at the top of all positive changes in curvature indicating its functional centrality in HGSOC.

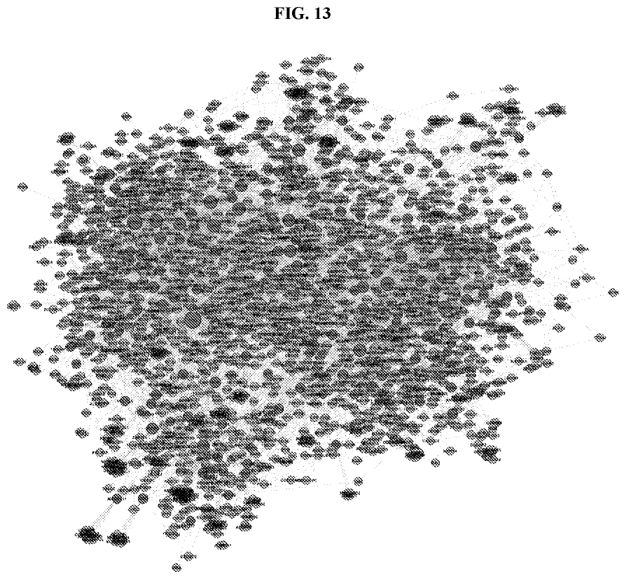

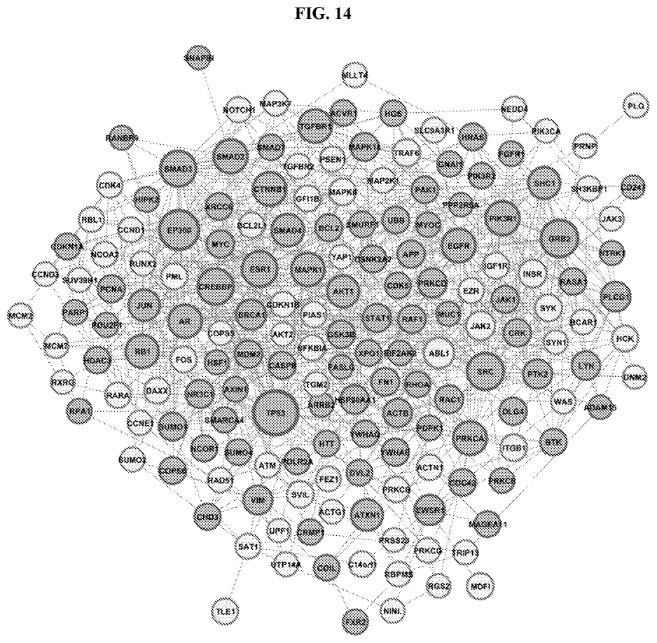

Substantial overlap in the top 50 (positive and negative) ranked genes was noted from all of the comparisons performed, resulting in 171 unique genes listed in Table 8 (FIGS. 13 and 14). The choice of selecting the top 50 genes was largely arbitrary with the following rationale. The assertion that critical genes may be identified as those exhibiting larger changes in curvature is supported by the theory, but curvature is a continuous variable with no obvious cutoff. Since there is also an exploratory component to this analysis, a cutoff that would yield a manageable set of genes that reasonably included the key influential players was desirable. Out of 3,489 genes in the network, this resulted in 50 (positive and negative) candidate genes. See FIG. 11 for a further sub-curvature analysis on the association between the highlighted candidate genes and survival.

TABLE 3
Changes in average scalar curvature based on risk (low vs high).

| Rank | Gene | Δ_{K}_{ᵣᵢₛₖ}> 0 | Gene | Δ_{K}_{ᵣᵢₛₖ}< 0 |
|---|---|---|---|---|
| 0 | TP53 | 0.208647 | CREBBP | −0.064223 |
| 1 | ATXN1 | 0.102823 | SHC1 | −0.031456 |
| 2 | EP300 | 0.044184 | PTK2 | −0.026202 |
| 3 | SMAD2 | 0.042756 | AR | −0.025316 |
| 4 | PIK3R1 | 0.037015 | MYC | −0.022608 |
| 5 | SRC | 0.033112 | JUN | −0.019546 |
| 6 | SMAD4 | 0.032177 | LYN | −0.011148 |
| 7 | RB1 | 0.031043 | YWHAQ | −0.010984 |
| 8 | ESR1 | 0.027914 | GSK38 | −0.009017 |
| 9 | PRKCA | 0.027253 | STAT1 | −0.008248 |
| 10 | CTNNB1 | 0.025121 | CDK5 | −0.007480 |
| 11 | GRB2 | 0.016848 | FN1 | −0.006947 |
| 12 | YWHAE | 0.015125 | COPS6 | −0.006251 |
| 13 | DLG4 | 0.014966 | SMAD3 | −0.006133 |
| 14 | PRKCD | 0.014742 | PAK1 | −0.006091 |
| 15 | ACTB | 0.013456 | MYOC | −0.005464 |
| 16 | EWSR1 | 0.012300 | SMURF1 | −0.005438 |
| 17 | TGFBR1 | 0.010799 | SUMO1 | −0.004455 |
| 18 | RAC1 | 0.008937 | PARP1 | −0.004274 |
| 19 | PLCG1 | 0.008423 | CRMP1 | −0.004271 |
| 20 | CHD3 | 0.007997 | HSF1 | −0.004155 |
| 21 | DVL2 | 0.007476 | HIPK2 | −0.004038 |
| 22 | BCL2 | 0.007009 | CDC42 | −0.004017 |
| 23 | RANBP9 | 0.006879 | POU2F1 | −0.003838 |
| 24 | MAPK1 | 0.006630 | ACVR1 | −0.003651 |
| 25 | POLRZA | 0.006468 | HTT | −0.003537 |
| 26 | CRK | 0.006375 | JAK1 | −0.003520 |
| 27 | APP | 0.006256 | PDPK1 | −0.003497 |
| 28 | PCNA | 0.005935 | PIK3R2 | −0.003423 |
| 29 | COIL | 0.005350 | FGFR1 | −0.003352 |
| 30 | MAPK14 | 0.005097 | CDKN1A | −0.003205 |
| 31 | NR3C1 | 0.004981 | MAGEA11 | −0.003165 |
| 32 | AKT1 | 0.004925 | GNAI1 | −0.003125 |
| 33 | EGFR | 0.004918 | PRKCE | −0.003090 |
| 34 | RHOA | 0.004635 | XPO1 | −0.002919 |
| 35 | RAF1 | 0.004159 | BTK | −0.002855 |
| 36 | SMAD7 | 0.004071 | MUC1 | −0.002814 |
| 37 | NCOR1 | 0.004038 | EIF2AK2 | −0.002807 |
| 38 | RASA1 | 0.003998 | CASPS | −0.002758 |
| 39 | FXR2 | 0.003879 | CSNK2A2 | −0.002717 |
| 40 | RPA1 | 0.003560 | MDM2 | −0.002710 |
| 41 | HRAS | 0.003525 | NTRK1 | −0.002636 |
| 42 | UBB | 0.003302 | ADAM15 | −0.002541 |
| 43 | BRCA3 | 0.003292 | FASLG | −0.002522 |
| 44 | SUMO4 | 0.003283 | VIM | −0.002436 |
| 45 | ARRB2 | 0.003248 | CD247 | −0.002372 |
| 46 | XRCC6 | 0.003065 | AXIN1 | −0.002333 |
| 47 | HGS | 0.003025 | SMARCA4 | −0.002256 |
| 48 | HDAC3 | 0.002965 | SNAPIN | −0.002246 |
| 49 | HSP90AA1 | 0.002924 | PPP2R5A | −0.002187 |

Top 50 genes ranked by positive (Δ_{K}_{ᵣᵢₛₖ} > 0) and negative (Δ_{K}_{ᵣᵢₛₖ} < 0) difference in average scalar curvature between low risk (n = 33) and high risk (n = 12) groups.

TABLE 4

| | | | | |
|---|---|---|---|---|
| ACTB | ACVR1 | ADAM15 | AKT1 | APP |
| AR | ARRB2 | ATXN1 | AXIN1 | BCL2 |
| BRCA1 | BTK | CASP8 | CD247 | CDC42 |
| CDK5 | CDKN1A | CHD3 | COIL | COPS6 |
| CREBBP | CRK | CRMP1 | CSNK2A2 | CTNNB1 |
| DLG4 | DVL2 | EGFR | EIF2AK2 | EP300 |
| ESR1 | EWSR1 | FASLG | FGFR1 | FN1 |
| FXR2 | GNAI1 | GRB2 | GSK3B | HDAC3 |
| HGS | HIPK2 | HRAS | HSF1 | HSP90AA1 |
| HTT | JAK1 | JUN | LYN | MAGEA11 |
| MAPK1 | MAPK14 | MDM2 | MUC1 | MYC |
| MYOC | NCOR1 | NR3C1 | NTRK1 | PAK1 |
| PARP1 | PCNA | PDPK1 | PIK3R1 | PIK3R2 |
| PLCG1 | POLR2A | POU2F1 | PPP2R5A | PRKCA |
| PRKCD | PRKCE | PTK2 | RAC1 | RAF1 |
| RANBP9 | RASA1 | RB1 | RHOA | RPA1 |
| SHC1 | SMAD2 | SMAD3 | SMAD4 | SMAD7 |
| SMARCA4 | SMURF1 | SNAPIN | SRC | STAT1 |
| SUMO1 | SUMO4 | TGFBR1 | TP53 | UBB |
| VIM | XPO1 | XRCC6 | YWHAE | YWHAQ |

TABLE 5
Changes in averae scalar curvature based on overall survival (OS)

| Rank | Gene | Δₙ_{_{OS}}> 0 | Gene | Δ_{K}_{_{OS}}< 0 |
|---|---|---|---|---|
| 0 | TP33 | 0.059206 | CTNNB1 | −0.029402 |
| 1 | SMAD3 | 0.055706 | CREBBP | −0.026285 |
| 2 | ATXN1 | 0.028886 | MYC | −0.024026 |
| 3 | EP300 | 0.028355 | PTK2 | −0.023118 |
| 4 | TGFBR1 | 0.017961 | AR | −0.021899 |
| 5 | AKT1 | 0.016810 | SHC1 | −0.018774 |
| 6 | JUN | 0.015342 | SMAD2 | −0.015914 |
| 7 | SRC | 0.013336 | RB1 | −0.015029 |
| 8 | ACTB | 0.013162 | VIM | −0.011736 |
| 9 | PONA | 0.010664 | PRKCA | −0.010209 |
| 10 | ESR1 | 0.010068 | SMAD4 | −0.009694 |
| 11 | ODKN1A | 0.009641 | MAPK1 | −0.008362 |
| 12 | RAC1 | 0.009197 | GRB2 | −0.008095 |
| 13 | CDKN1B | 0.008439 | SVIL | −0.005490 |
| 14 | PRKCD | 0.008138 | APP | −0.005477 |
| 15 | HSP90AA1 | 0.007070 | SMARCAA | −0.005419 |
| 16 | CONE1 | 0.006931 | PARP1 | −0.005321 |
| 17 | STAT1 | 0.006750 | FN1 | −0.005122 |
| 18 | COPS6 | 0.006061 | PIK3R2 | −0.004975 |
| 19 | MAPK14 | 0.005247 | CRMP1 | −0.004917 |
| 20 | MDFI | 0.005216 | MAPKS | −0.004334 |
| 21 | SMURF1 | 0.005144 | ITGB1 | −0.004226 |
| 22 | ODKS | 0.004177 | HTT | −0.004059 |
| 23 | ACTN1 | 0.004095 | HSF1 | −0.004010 |
| 24 | YWHABE | 0.003982 | INSR | −0.003442 |
| 25 | DLG4 | 0.003779 | LYN | −0.003200 |
| 26 | C14orf1 | 0.003745 | BTK | −0.003056 |
| 27 | JAK1 | 0.003667 | JAK3 | −0.003018 |
| 28 | PIAS1 | 0.003654 | YAP1 | −0.002875 |
| 29 | FOS | 0.003110 | GSK3B | −0.002822 |
| 30 | PLCG1 | 0.003333 | ATM | −0.002806 |
| 31 | CHD3 | 0.003285 | YWHAQ | −0.002480 |
| 32 | EWSR1 | 0.003279 | BCL2 | −0.002338 |
| 33 | PIK3R1 | 0.003136 | WAS | −0.002229 |
| 34 | NEKB1A | 0.003024 | PPP2RSA | −0.002188 |
| 35 | PAIL | 0.002953 | MUC1 | −0.002134 |
| 36 | PRAP | 0.002944 | SUV39H1 | −0.002114 |
| 37 | RBPMS | 0.002917 | TGEBR2 | −0.002039 |
| 38 | COND3 | 0.002861 | ADAM15 | −0.001992 |
| 39 | RUNX2 | 0.002838 | MAGEA11 | −0.001903 |
| 40 | MAPZK1 | 0.002709 | POU2F1 | −0.001759 |
| 41 | PSEN1 | 0.002696 | SYN1 | −0.001755 |
| 42 | BRCA1 | 0.002660 | PRKCG | −0.001745 |
| 43 | XPO1 | 0.002648 | RGS2 | −0.001736 |
| 44 | DVL2 | 0.002554 | DNM2 | −0.001634 |
| 45 | CRK | 0.002550 | PAK1 | −0.001629 |
| 46 | TRAF6 | 0.002531 | FEZ1 | −0.001536 |
| 47 | MCM7 | 0.002298 | JAK2 | −0.001488 |
| 48 | NEDD4 | 0.002287 | UPF1 | −0.001487 |
| 49 | RAD51 | 0.002286 | MDM2 | −0.001449 |

Top 50 genes ranked by positive (Δκ_{OS}>0) and negative (Δκ_{OS}<0) difference in average scalar curvature between alive (n=13) and dead (n=32) cohorts (at last follow-up).

TABLE 6
Changes in average scalar curvature based on
sample type (primary (P) vs metastasis (M))

| Rank | Gene | Δ_{K}_{_{PM}}> 0 | Gene | Δ_{K}_{_{PM}}< 0 |
|---|---|---|---|---|
| 0 | TP53 | 0.077029 | ESR1 | −0.036440 |
| 1 | GRB2 | 0.052007 | SMAD3 | −0.021618 |
| 2 | ATXN1 | 0.047675 | CDKN1A | −0.020347 |
| 3 | PRKCA | 0.042002 | JUN | −0.015349 |
| 4 | CREBBP | 0.026753 | SRC | −0.013494 |
| 5 | SMAD2 | 0.019927 | LYN | −0.011455 |
| 6 | AKT1 | 0.017599 | MDFI | −0.010594 |
| 7 | TGFBR1 | 0.012515 | EGFR | −0.008569 |
| 8 | SMAD4 | 0.011964 | MAPK14 | −0.008072 |
| 9 | CTNNB1 | 0.010307 | CONE1 | −0.007612 |
| 10 | EWSR1 | 0.010187 | GSK3B | −0.006226 |
| 11 | VIM | 0.009960 | RB1 | −0.005719 |
| 12 | EP300 | 0.009848 | RUNX2 | −0.005594 |
| 13 | APP | 0.007988 | COND3 | −0.005412 |
| 14 | HSP90AA1 | 0.007883 | MYOC | −0.005080 |
| 15 | MYC | 0.007121 | JAK2 | −0.004539 |
| 16 | PTK2 | 0.005757 | CDKN1B | −0.004367 |
| 17 | HGS | 0.005709 | DAXX | −0.004313 |
| 18 | YWHAE | 0.005597 | RBPMS | −0.004050 |
| 19 | DLG4 | 0.005540 | PIK3R2 | −0.004026 |
| 20 | SVIL | 0.005213 | MAP3K7 | −0.003988 |
| 21 | COIL | 0.005048 | PRKCD | −0.003983 |
| 22 | PIKBR1 | 0.004457 | MDM2 | −0.003608 |
| 23 | ITGB1 | 0.004411 | NCOA2 | −0.003404 |
| 24 | STAT1 | 0.004319 | RAF1 | −0.003399 |
| 25 | SLO9A3R1 | 0.004226 | RAC1 | −0.003303 |
| 26 | MAPK1 | 0.004204 | AKT2 | −0.003245 |
| 27 | ABL1 | 0.003941 | PLCG1 | −0.002819 |
| 28 | CDK5 | 0.003806 | HRAS | −0.002807 |
| 29 | BRCA1 | 0.003210 | IGF1R | −0.002795 |
| 30 | ACTN1 | 0.003170 | EZR | −0.002769 |
| 31 | RANBP9 | 0.003063 | MLUT4 | −0.002732 |
| 32 | PRKOG | 0.002898 | PRKCB | −0.002682 |
| 33 | GFI1B | 0.002779 | POU2F1 | −0.002679 |
| 34 | TLE1 | 0.002757 | FASLG | −0.002679 |
| 35 | CHD3 | 0.002688 | JAK1 | −0.002676 |
| 36 | CCND1 | 0.002670 | GNAI1 | −0.002552 |
| 37 | SYK | 0.002652 | PCNA | −0.002510 |
| 38 | MAPK8 | 0.002550 | PLG | −0.002244 |
| 39 | DVL2 | 0.002504 | FN1 | −0.002154 |
| 40 | YAP1 | 0.002367 | XPO1 | −0.002072 |
| 41 | BCL2 | 0.002288 | ACTB | −0.002056 |
| 42 | HTT | 0.002270 | CDC42 | −0.002046 |
| 43 | POLR2A | 0.002198 | SHC1 | −0.001900 |
| 44 | TRIP13 | 0.002189 | SUMO4 | −0.001896 |
| 45 | ATM | 0.002159 | JAK3 | −0.001888 |
| 46 | CRMP1 | 0.002127 | COPS5 | −0.001875 |
| 47 | ACTG1 | 0.002125 | CD247 | −0.001809 |
| 48 | C14orf1 | 0.002115 | PIK3CA | −0.001768 |
| 49 | SUMO2 | 0.001974 | RXRG | −0.001731 |

Top 50 genes ranked by positive (Δκ_{PM}>0) and negative (Δκ_{PM}<0) difference in average scalar curvature between P (n=13) and M (n=32) groups.

TABLE 7
Changes in average scalar curvature with
respect to the reference topology

| Rank | Gene | Δ_{K}_{_{ref}}> 0 | Gene | Δ_{K}_{_{ref}}< 0 |
|---|---|---|---|---|
| 0 | TP53 | 0.143999 | SRC | −0.070044 |
| 1 | EP300 | 0.089752 | ATXN1 | −0.041866 |
| 2 | AR | 0.055047 | PTK2 | −0.040546 |
| 3 | TGFBR1 | 0.050407 | MYC | −0.032654 |
| 4 | MAPK1 | 0.041837 | LYN | −0.026977 |
| 5 | ESR1 | 0.025883 | SHC1 | −0.021819 |
| 6 | PIK3R1 | 0.024060 | GSK3B | −0.017265 |
| 7 | SMAD3 | 0.023103 | JUN | −0.015936 |
| 8 | RB1 | 0.021967 | PCNA | −0.015054 |
| 9 | EWSR1 | 0.020344 | PLCG1 | −0.014421 |
| 10 | SMAD4 | 0.019587 | MAPK14 | −0.014055 |
| 11 | SMAD2 | 0.019196 | CDKN1A | −0.011164 |
| 12 | CREBBP | 0.018241 | YWHAQ | −0.008725 |
| 13 | ABL1 | 0.016583 | MDM2 | −0.007339 |
| 14 | CSNK2A2 | 0.012132 | NCOA2 | −0.007238 |
| 15 | DLG4 | 0.011312 | CCNE1 | −0.006786 |
| 16 | CTNNB1 | 0.010916 | CDKN1B | −0.006716 |
| 17 | BRCA1 | 0.010490 | HCK | −0.006688 |
| 18 | EGFR | 0.009949 | PAK1 | −0.006402 |
| 19 | YWHAE | 0.009881 | DAXX | −0.006289 |
| 20 | GFI1B | 0.009811 | HSF1 | −0.006140 |
| 21 | ACTB | 0.009696 | BCL2L1 | −0.005700 |
| 22 | RAC1 | 0.009329 | RBL1 | −0.005566 |
| 23 | MAGEA11 | 0.009010 | MDFI | −0.005182 |
| 24 | BTK | 0.008727 | STAT1 | −0.004959 |
| 25 | XRCC6 | 0.007943 | ACVR1 | −0.004607 |
| 26 | UBB | 0.007720 | SUMO1 | −0.004323 |
| 27 | AKT1 | 0.007642 | RANBP9 | −0.004323 |
| 28 | CHD3 | 0.007446 | COPS5 | −0.004273 |
| 29 | TLE1 | 0.007259 | CDK5 | −0.004024 |
| 30 | SAT1 | 0.006644 | MYOC | −0.003970 |
| 31 | JAK2 | 0.006454 | FN1 | −0.003823 |
| 32 | DVL2 | 0.006230 | PARP1 | −0.003753 |
| 33 | SYK | 0.006148 | CDK4 | −0.003741 |
| 34 | NOTCH1 | 0.005997 | CCND3 | −0.003324 |
| 35 | POLR2A | 0.005812 | COPS6 | −0.003277 |
| 36 | NCOR1 | 0.005568 | SMURF1 | −0.003160 |
| 37 | HSP90AA1 | 0.005143 | HIPK2 | −0.003070 |
| 38 | INSR | 0.005123 | POU2F1 | −0.003066 |
| 39 | CRK | 0.004940 | XPO1 | −0.003044 |
| 40 | PRKCB | 0.004893 | TGM2 | −0.003020 |
| 41 | BCAR1 | 0.004520 | FGFR1 | −0.002781 |
| 42 | HTT | 0.004499 | PRNP | −0.002770 |
| 43 | BCL2 | 0.004241 | MUC1 | −0.002713 |
| 44 | SH3KBP1 | 0.003904 | TRAF6 | −0.002684 |
| 45 | UTP14A | 0.003815 | YAP1 | −0.002674 |
| 46 | PRKCD | 0.003753 | MCM2 | −0.002534 |
| 47 | RASA1 | 0.003679 | RUNX2 | −0.002534 |
| 48 | RARA | 0.003616 | PRSS23 | −0.002533 |
| 49 | FXR2 | 0.003610 | NINL | −0.002514 |

Top 50 genes ranked by positive (Δκ_{ref}>0) and negative (Δκ_{ref}<0) difference between average HGSOC scalar curvature (n=45) κ_{HGS} and the scalar curvature of the reference topology κₜₒₚ. Δκ_{ref}=κ_{HGS}−κₜₒₚ.

TABLE 8
Intersection of 171 top ranked candidate genes listed alphabetically

ABL1
ACTB
ACTG1
ACTN1
ACVR1
ADAM15
AKT1
AKT2
APP
AR
ARRB2
ATM
ATXN1
AXIN1
BCAR1
BCL2
BCL2L1
BRCA1
BTK
C14orf1
CASP8
CCND1
CCND3
CCNE1
CD247
CDC42
CDK4
CDK5
CDKN1A
CDKN1B
CHD3
COIL
COPS5
COPS6
CREBBP
CRK
CRMP1
CSNK2A2
CTNNB1
DAXX
DLG4
DNM2
DVL2
EGFR
EIF2AK2
EP300
ESR1
EWSB1
EZR
FASLG
FEZ1
FGFR1
FN1
FOS
FXR2
GFI1B
GNAI1
GRB2
GSK3B
HCK
HDAC3
HGS
HIPK2
HRAS
HSF1
HSP90AA1
HTT
IGF1R
INSR
ITGB1
JAK1
JAK2
JAK3
JUN
LYN
MAGEA11
MAP2K1
MAP3K7
MAPK1
MAPK14
MAPK8
MCM2
MCM7
MDFI
MDM2
MLLT4
MUC1
MYC
MYOC
NCOA2
NCOR1
NEDD4
NEKB1A
NINL
NOTCH1
NR3C1
NTRK1
PAK1
PARP1
PCNA
PDPK1
PIAS1
PIK3CA
PIK3R1
PIK3R2
PLCG1
PLG
PML
POLR2A
POU2F1
PPP2RSA
PRKCA
PRKCB
PRKCD
PRKCE
PRKCG
PRNP
PRSS23
PSEN1
PTK2
RAC1
RAD51
RAF1
RANBP9
RARA
RASA1
RB1
RBL1
RBPMS
RGS2
RHOA
RPA1
RUNX2
RXRG
SAT1
SH3KBP1
SHC1
SLC9A3R1
SMAD2
SMAD3
SMAD4
SMAD7
SMARCA4
SMURF1
SNAPIN
SRC
STAT1
SUMO1
SUMO2
SUMO4
SUV39H1
SVIL
SYK
SYN1
TGFBR1
TGFBR2
TGM2
TLE1
TP53
TRAF6
TRIP13
UBB
UPF1
UTP14A
VIM
WAS
XPO1
XRCC6
YAP1
YWHAE
YWHAQ

TABLE 9
100 identified candidate genes based on risk listed alphabetically

| | | | | |
|---|---|---|---|---|
| ACTB | ACVR1 | ADAM15 | AKT1 | APP |
| AR | ARRB2 | ATXN1 | AXIN1 | BCL2 |
| BRCA1 | BTK | CASP8 | CD247 | CDC42 |
| CDK5 | CDKN1A | CHD3 | COIL | COPS6 |
| CREBBP | CRK | CRMP1 | CSNK2A2 | CTNNB1 |
| DLG4 | DVL2 | EGFR | EIF2AK2 | EP300 |
| ESR1 | EWSR1 | FASLG | FGFR1 | FN1 |
| FXR2 | GNAI1 | GRB2 | GSK3B | HDAC3 |
| HGS | HIPK2 | HRAS | HSF1 | HSP90AA1 |
| HTT | JAK1 | JUN | LYN | MAGEA11 |
| MAPK1 | MAPK14 | MDM2 | MUC1 | MYC |
| MYOC | NCOR1 | NR3C1 | NTRK1 | PAK1 |
| PARP1 | PCNA | PDPK1 | PIK3R1 | PIK3R2 |
| PLCG1 | POLR2A | POU2F1 | PPP2R5A | PRKCA |
| PRKCD | PRKCE | PTK2 | RAC1 | RAF1 |
| RANBP9 | RASA1 | RB1 | RHOA | RPA1 |
| SHC1 | SMAD2 | SMAD3 | SMAD4 | SMAD7 |
| SMARCA4 | SMURF1 | SNAPIN | SRC | STAT1 |
| SUMO1 | SUMO4 | TGFBR1 | TP53 | UBB |
| VIM | XPO1 | XRCC6 | YWHAE | YWHAQ |

### Example 4: Relationship Between Total Curvature and Genomic Features

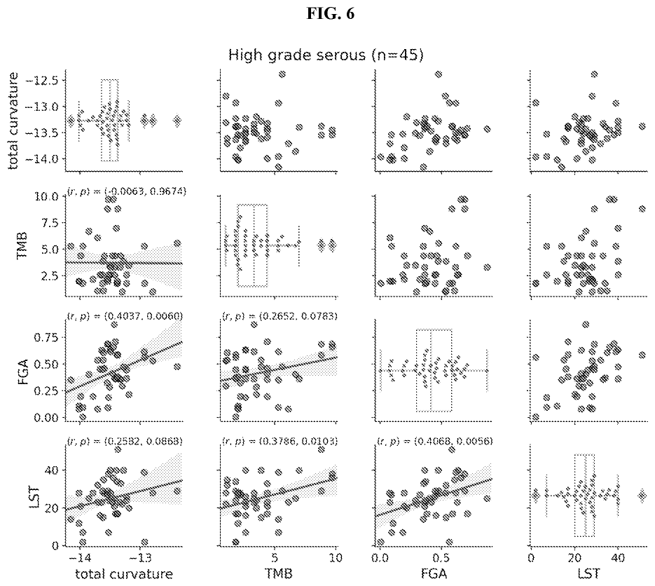

Lastly, the relationship between total curvature and genomic features (TMB, FGA, LST) was explored. Linear regression analysis and Pearson correlation (r) with p-values were used to assess the correlation between total curvature and each of the clinical features (TMB: p=0.9674; FGA: p=0.0059; LST: p=0.0867). This analysis suggests that total curvature is significantly correlated with FGA. FGA is a surrogate measure of CN changes and the curvature measures dysregulation of the CN-weighted network. However, total curvature yields high and low risk groups with a significant difference in survival whereas FGA does not. The difference is that total curvature accounts for an extra level of information, namely the connectivity, which is not evident from CNAs alone. This is compelling evidence that network dysregulation, as measured by curvature, has the potential to provide critical insight for analyzing immune response. More samples are needed to verify this result but it is interesting to note that further investigation into FGA as a potential biomarker for survival in HGSOC has been proposed. Linear regression plots on the HGSOC cohort (n=45) are shown in FIG. 6.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

### Computing and Network Environment

Various operations described herein can be implemented on computer systems. FIG. 4 shows a simplified block diagram of a representative server system 400, client computer system 414, and network 426 usable to implement certain embodiments of the present disclosure. In various embodiments, server system 400 or similar systems can implement services or servers described herein or portions thereof. Client computer system 414 or similar systems can implement clients described herein. The system 100 described herein can be similar to the server system 400. Server system 400 can have a modular design that incorporates a number of modules 402 (e.g., blades in a blade server embodiment); while two modules 402 are shown, any number can be provided. Each module 4s02 can include processing unit(s) 404 and local storage 406.

Processing unit(s) 404 can include a single processor, which can have one or more cores, or multiple processors. In some embodiments, processing unit(s) 404 can include a general-purpose primary processor as well as one or more special-purpose co-processors such as graphics processors, digital signal processors, or the like. In some embodiments, some or all processing units 404 can be implemented using customized circuits, such as application specific integrated circuits (ASICs) or field programmable gate arrays (FPGAs). In some embodiments, such integrated circuits execute instructions that are stored on the circuit itself. In other embodiments, processing unit(s) 404 can execute instructions stored in local storage 406. Any type of processors in any combination can be included in processing unit(s) 404.

Local storage 406 can include volatile storage media (e.g., DRAM, SRAM, SDRAM, or the like) and/or non-volatile storage media (e.g., magnetic or optical disk, flash memory, or the like). Storage media incorporated in local storage 406 can be fixed, removable or upgradeable as desired. Local storage 406 can be physically or logically divided into various subunits such as a system memory, a read-only memory (ROM), and a permanent storage device. The system memory can be a read-and-write memory device or a volatile read-and-write memory, such as dynamic random-access memory. The system memory can store some or all of the instructions and data that processing unit(s) 404 need at runtime. The ROM can store static data and instructions that are needed by processing unit(s) 404. The permanent storage device can be a non-volatile read-and-write memory device that can store instructions and data even when module 402 is powered down. The term “storage medium” as used herein includes any medium in which data can be stored indefinitely (subject to overwriting, electrical disturbance, power loss, or the like) and does not include carrier waves and transitory electronic signals propagating wirelessly or over wired connections.

In some embodiments, local storage 406 can store one or more software programs to be executed by processing unit(s) 404, such as an operating system and/or programs implementing various server functions such as functions of the system 100 of FIG. 1 or any other system described herein, or any other server(s) associated with system 100 or any other system described herein.

“Software” refers generally to sequences of instructions that, when executed by processing unit(s) 404 cause server system 400 (or portions thereof) to perform various operations, thus defining one or more specific machine embodiments that execute and perform the operations of the software programs. The instructions can be stored as firmware residing in read-only memory and/or program code stored in non-volatile storage media that can be read into volatile working memory for execution by processing unit(s) 404. Software can be implemented as a single program or a collection of separate programs or program modules that interact as desired. From local storage 406 (or non-local storage described below), processing unit(s) 404 can retrieve program instructions to execute and data to process in order to execute various operations described above. s

In some server systems 400, multiple modules 402 can be interconnected via a bus or other interconnect 408, forming a local area network that supports communication between modules 402 and other components of server system 400. Interconnect 408 can be implemented using various technologies including server racks, hubs, routers, etc.

A wide area network (WAN) interface 410 can provide data communication capability between the local area network (interconnect 408) and the network 426, such as the Internet. Technologies can be used, including wired (e.g., Ethernet, IEEE 802.3 standards) and/or wireless technologies (e.g., Wi-Fi, IEEE 802.11 standards).

In some embodiments, local storage 406 is intended to provide working memory for processing unit(s) 404, providing fast access to programs and/or data to be processed while reducing traffic on interconnect 408. Storage for larger quantities of data can be provided on the local area network by one or more mass storage subsystems 412 that can be connected to interconnect 408. Mass storage subsystem 412 can be based on magnetic, optical, semiconductor, or other data storage media. Direct attached storage, storage area networks, network-attached storage, and the like can be used. Any data stores or other collections of data described herein as being produced, consumed, or maintained by a service or server can be stored in mass storage subsystem 412. In some embodiments, additional data storage resources may be accessible via WAN interface 410 (potentially with increased latency).

Server system 400 can operate in response to requests received via WAN interface 410. For example, one of modules 402 can implement a supervisory function and assign discrete tasks to other modules 402 in response to received requests. Work allocation techniques can be used. As requests are processed, results can be returned to the requester via WAN interface 410. Such operation can generally be automated. Further, in some embodiments, WAN interface 410 can connect multiple server systems 400 to each other, providing scalable systems capable of managing high volumes of activity. Other techniques for managing server systems and server farms (collections of server systems that cooperate) can be used, including dynamic resource allocation and reallocation.

Server system 400 can interact with various user-owned or user-operated devices via a wide-area network such as the Internet. An example of a user-operated device is shown in FIG. 4 as client computing system 414. Client computing system 414 can be implemented, for example, as a consumer device such as a smartphone, other mobile phone, tablet computer, wearable computing device (e.g., smart watch, eyeglasses), desktop computer, laptop computer, and so on.

For example, client computing system 414 can communicate via WAN interface 410. Client computing system 414 can include computer components such as processing unit(s) 416, storage device 418, network interface 420, user input device 422, and user output device 424. Client computing system 414 can be a computing device implemented in a variety of form factors, such as a desktop computer, laptop computer, tablet computer, smartphone, other mobile computing device, wearable computing device, or the like.

Processor 416 and storage device 418 can be similar to processing unit(s) 404 and local storage 406 described above. Suitable devices can be selected based on the demands to be placed on client computing system 414; for example, client computing system 414 can be implemented as a “thin” client with limited processing capability or as a high-powered computing device. Client computing system 414 can be provisioned with program code executable by processing unit(s) 416 to enable various interactions with server system 400.

Network interface 420 can provide a connection to the network 426, such as a wide area network (e.g., the Internet) to which WAN interface 410 of server system 400 is also connected. In various embodiments, network interface 420 can include a wired interface (e.g., Ethernet) and/or a wireless interface implementing various RF data communication standards such as Wi-Fi, Bluetooth, or cellular data network standards (e.g., 3G, 4G, LTE, etc.).

User input device 422 can include any device (or devices) via which a user can provide signals to client computing system 414; client computing system 414 can interpret the signals as indicative of particular user requests or information. In various embodiments, user input device 422 can include any or all of a keyboard, touch pad, touch screen, mouse or other pointing device, scroll wheel, click wheel, dial, button, switch, keypad, microphone, and so on.

User output device 424 can include any device via which client computing system 414 can provide information to a user. For example, user output device 424 can include a display to display images generated by or delivered to client computing system 414. The display can incorporate various image generation technologies, e.g., a liquid crystal display (LCD), light-emitting diode (LED) including organic light-emitting diodes (OLED), projection system, cathode ray tube (CRT), or the like, together with supporting electronics (e.g., digital-to-analog or analog-to-digital converters, signal processors, or the like). Some embodiments can include a device such as a touchscreen that functions as both input and output device. In some embodiments, other user output devices 424 can be provided in addition to or instead of a display. Examples include indicator lights, speakers, tactile “display” devices, printers, and so on.

Some embodiments include electronic components, such as microprocessors, storage and memory that store computer program instructions in a computer readable storage medium. Many of the features described in this specification can be implemented as processes that are specified as a set of program instructions encoded on a computer readable storage medium. When these program instructions are executed by one or more processing units, they cause the processing unit(s) to perform various operations indicated in the program instructions. Examples of program instructions or computer code include machine code, such as is produced by a compiler, and files including higher-level code that are executed by a computer, an electronic component, or a microprocessor using an interpreter. Through suitable programming, processing unit(s) 404 and 416 can provide various functionality for server system 400 and client computing system 414, including any of the functionality described herein as being performed by a server or client, or other functionality.

It will be appreciated that server system 400 and client computing system 414 are illustrative and that variations and modifications are possible. Computer systems used in connection with embodiments of the present disclosure can have other capabilities not specifically described here. Further, while server system 400 and client computing system 414 are described with reference to particular blocks, it is to be understood that these blocks are defined for convenience of description and are not intended to imply a particular physical arrangement of component parts. For instance, different blocks can be but need not be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments of the present disclosure can be realized in a variety of apparatus including electronic devices implemented using any combination of circuitry and software.

While the disclosure has been described with respect to specific embodiments, one skilled in the art will recognize that numerous modifications are possible. Embodiments of the disclosure can be realized using a variety of computer systems and communication technologies including but not limited to specific examples described herein. Embodiments of the present disclosure can be realized using any combination of dedicated components and/or programmable processors and/or other programmable devices. The various processes described herein can be implemented on the same processor or different processors in any combination. Where components are described as being configured to perform certain operations, such configuration can be accomplished, e.g., by designing electronic circuits to perform the operation, by programming programmable electronic circuits (such as microprocessors) to perform the operation, or any combination thereof. Further, while the embodiments described above may make reference to specific hardware and software components, those skilled in the art will appreciate that different combinations of hardware and/or software components may also be used and that particular operations described as being implemented in hardware might also be implemented in software or vice versa.

Computer programs incorporating various features of the present disclosure may be encoded and stored on various computer readable storage media; suitable media include magnetic disk or tape, optical storage media such as compact disk (CD) or DVD (digital versatile disk), flash memory, and other non-transitory media. Computer readable media encoded with the program code may be packaged with a compatible electronic device, or the program code may be provided separately from electronic devices (e.g., via Internet download or as a separately packaged computer-readable storage medium).

Thus, although the disclosure has been described with respect to specific embodiments, it will be appreciated that the disclosure is intended to cover all modifications and equivalents within the scope of the following claims.

Aspects can be combined and it will be readily appreciated that features described in the context of one aspect can be combined with other aspects. Aspects can be implemented in any convenient form. For example, by appropriate computer programs, which may be carried on appropriate carrier media (computer readable media), which may be tangible carrier media (e.g. disks) or intangible carrier media (e.g. communications signals). Aspects may also be implemented using a suitable apparatus, which can take the form of one or more programmable computers running computer programs arranged to implement the aspect. As used in the specification and in the claims, the singular form of ‘a’, ‘an’, and ‘the’ include plural referents unless the context clearly dictates otherwise.

1. A method for selecting a patient suffering from or diagnosed with ovarian cancer for treatment with an immune checkpoint inhibitor therapy comprising:
constructing a network comprising a plurality of nodes and edges representing a plurality of gene pathways, wherein each node of the network is a gene that is a component of a gene pathway and wherein each node is associated with a copy number alteration (CNA) metric, wherein the CNA metric for each node is based on genomic sequence data corresponding to at least one tumor of the patient;
computing for the network an edge curvature for each of the plurality of edges;
computing a node curvature for each of the plurality of nodes based on the edge curvatures of edges incident to each of the plurality of nodes;
computing a total curvature metric of the network by determining a net node curvature over the plurality of nodes of the network; and
selecting the patient for treatment with an immune checkpoint inhibitor therapy when the patient exhibits a total curvature metric satisfies a predetermined threshold.

2. The method of claim 1, wherein the ovarian cancer is high grade serous ovarian cancer.

3. The method of claim 1, wherein the genomic sequence data is obtained by sequencing circulating tumor DNA (ctDNA) or sequencing DNA from a biopsied tumor isolated from the patient.

4. The method of claim 3, wherein the genomic sequence data is obtained via whole exome sequencing or targeted exome sequencing.

5. The method of claim 1, wherein the network is constructed using a protein-protein interactome (PPI) database.

6. The method of claim 5, wherein the PPI database is Human Reference Protein Database (HPRD), Human Reference Interactome (HuRI), or Search Tool for the Retrieval of Interacting Genes/Proteins (STRING).

7. The method of claim 1, wherein the immune checkpoint inhibitor therapy comprises one or more of an anti-PD-1 antibody, an anti-PD-L1 antibody, an anti-PD-L2 antibody, an anti-CTLA-4 antibody, an anti-TIM3 antibody, an anti-TIGIT antibody, an anti-VISTA antibody, an anti-B7-H3 antibody, an anti-BTLA antibody, an anti-CD73 antibody, or an anti-LAG-3 antibody.

8. The method of claim 1, wherein the immune checkpoint inhibitor therapy comprises one or more of pembrolizumab, nivolumab, cemiplimab, atezolizumab, avelumab, durvalumab, ipilimumab, tremelimumab, ticlimumab, JTX-4014, Spartalizumab (PDR001), Camrelizumab (SHR1210), Sintilimab (IB1I308), Tislelizumab (BGB-A317), Toripalimab (JS 001), Dostarlimab (TSR-042, WBP-285), INCMGA00012 (MGA012), AMP-224, AMP-514, KN035, CK-301, AUNP12, CA-170, or BMS-986189.

9. The method of claim 1, wherein the plurality of nodes of the network comprises ACTB, ACVR1, ADAM15, AKT1, APP, AR, ARRB2, ATXN1, AXIN1, BCL2, BRCA1, BTK, CASP8, CD247, CDC42, CDK5, CDKN1A, CHD3, COIL, COPS6, CREBBP, CRK, CRMP1, CSNK2A2, CTNNB1, DLG4, DVL2, EGFR, EIF2AK2, EP300, ESR1, EWSR1, FASLG, FGFR1, FN1, FXR2, GNAI1, GRB2, GSK3B, HDAC3, HGS, HIPK2, HRAS, HSF1, HSP90AA1, HTT, JAK1, JUN, LYN, MAGEA11, MAPK1, MAPK14, MDM2, MUC1, MYC, MYOC, NCOR1, NR3C1, NTRK1, PAK1, PARP1, PCNA, PDPK1, PIK3R1, PIK3R2, PLCG1, POLR2A, POU2F1, PPP2R5A, PRKCA, PRKCD, PRKCE, PTK2, RAC1, RAF1, RANBP9, RASA1, RB1, RHOA, RPA1, SHC1, SMAD2, SMAD3, SMAD4, SMAD7, SMARCA4, SMURF1, SNAPIN, SRC, STAT1, SUMO1, SUMO4, TGFBR1, TP53, UBB, VIM, XPO1, XRCC6, YWHAE, and YWHAQ.

10. The method of claim 1, wherein the plurality of nodes of the network comprises ABL1, ACTB, ACTG1, ACTN1, ACVR1, ADAM15, AKT1, AKT2, APP, ΔR, ΔRRB2, ATM, ATXN1, AXIN1, BCAR1, BCL2, BCL2L1, BRCA1, BTK, C14orfl, CASP8, CCND1, CCND3, CCNE1, CD247, CDC42, CDK4, CDK5, CDKN1A, CDKN1B, CHD3, COIL, COPS5, COPS6, CREBBP, CRK, CRMP1, CSNK2A2, CTNNB1, DAXX, DLG4, DNM2, DVL2, EGFR, EIF2AK2, EP300, ESR1, EWSR1, EZR, FASLG, FEZ1, FGFR1, FN1, FOS, FXR2, GFI1B, GNAI1, GRB2, GSK3B, HCK, HDAC3, HGS, HIPK2, HRAS, HSF1, HSP90AA1, HTT, IGF1R, INSR, ITGB1, JAK1, JAK2, JAK3, JUN, LYN, MAGEA11, MAP2K1, MAP3K7, MAPK1, MAPK14, MAPK8, MCM2, MCM7, MDF1, MDM2, MLLT4, MUC1, MYC, MYOC, NCOA2, NCOR1, NEDD4, NFKBIA, NINL, NOTCH1, NR3C1, NTRK1, PAK1, PARP1, PCNA, PDPK1, PIAS1, PIK3CA, PIK3R1, PIK3R2, PLCG1, PLG, PML, POLR2A, POU2F1, PPP2R5A, PRKCA, PRKCB, PRKCD, PRKCE, PRKCG, PRNP, PRSS23, PSEN1, PTK2, RAC1, RAD51, RAF1, RANBP9, RARA, RASA1, RB1, RBL1, RBPMS, RGS2, RHOA, RPA1, RUNX2, RXRG, SAT1, SH3KBP1, SHC1, SLC9A3R1, SMAD2, SMAD3, SMAD4, SMAD7, SMARCA4, SMURF1, SNAPIN, SRC, STAT1, SUMO1, SUMO2, SUMO4, SUV39H1, SVIL, SYK, SYN1, TGFBR1, TGFBR2, TGM2, TLE1, TP53, TRAF6, TRIP13, UBB, UPF1, UTP14A, VIM, WAS, XPO1, XRCC6, YAP1, YWHAE, and YWHAQ.

11. The method of claim 1, wherein the patient is LST-high.

12. The method of claim 1, wherein the patient exhibits high tumor mutation burden (TMB) or low TMB.

13. The method of claim 1, wherein the patient suffers from stage III or stage IV ovarian cancer.

14. The method of claim 1, wherein the ovarian cancer is metastatic or primary.

15. The method of claim 1, wherein the edge curvature comprises an Ollivier-Ricci curvature.

16. The method of claim 1, wherein the edge curvature is further based on a weighted hop distance between two nodes associated with a respective edge, wherein the weighted hop distance is determined using node weights associated with the CNA metrics for the two nodes associated with the respective edge.

17. The method of claim 16, wherein the weighted hop distance is indicative of a likelihood of interaction between the genes associated with the two nodes associated with the respective edge.

18. The method of claim 1, wherein the node curvature comprises a scalar curvature for a corresponding gene determined from a weighted sum of curvatures on all edges incident to the corresponding gene.

19. The method of claim 1, wherein the total curvature metric comprises a net scalar curvature summed over all nodes of the network.

20. A system, comprising:
one or more processors coupled to a non-transitory memory, the one or more processors configured to:
construct a network comprising a plurality of nodes and edges representing a plurality of gene pathways, wherein each node of the network is a gene that is a component of a gene pathway and wherein each node is associated with a copy number alteration (CNA) metric, wherein the CNA metric for each node is based on genomic sequence data corresponding to at least one tumor of the patient;
compute for the network an edge curvature for each of the plurality of edges;
compute a node curvature for each of the plurality of nodes based on the edge curvatures of edges incident to each of the plurality of nodes;
compute a total curvature metric of the network by determining a net node curvature over the plurality of nodes of the network; and
select the patient for treatment with an immune checkpoint inhibitor therapy when the patient exhibits a total curvature metric that is higher than a predetermined threshold.
